(19) United States
(12) Patent Application Publication
(10) Pub. No.: US 2010/0267784 A1
(43) Pub. Date: Oct. 21, 2010

(54) NOVEL 4,5-DIHYDROISOXAZOLES WITH ESTROGENIC ACTIVITY

(76) Inventors: Juha Pulkkinen, Kuopio (FI); Paavo Honkakoski, Kuopio (FI); Mikael Peräkylä, Kuopio (FI); Istvan Berczi, Lorette (CA); Reino Laatikainen, Kuopio (FI)

Correspondence Address: BIRCH STEWART KOLASCH & BIRCH, PO BOX 747, FALLS CHURCH, VA, 22040-0747, US

(21) Appl. No.: 12/744,256

(22) PCT Filed: Nov. 21, 2008
(86) PCT No.: PCT/FI2008/050675

§ 371 Date: Jun. 4, 2010

(30) Foreign Application Priority Data
Nov. 23, 2007 (FI) 20070897

(51) Int. Cl. A61K 31/42; C07D 261/04; A61P 19/08; A61P 19/10; A61P 9/00; A61P 1/00; A61P 3/04; A61P 15/00; A61P 25/22; A61P 25/24; A61P 9/12

(52) U.S. Cl. 514/378; 548/240

(57) ABSTRACT

This invention relates to novel 4,5-dihydroisoxazoles of formula (I), to their use as estrogen receptor modulators, and to methods of their preparation.

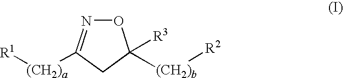

### FIELD OF THE INVENTION

This invention relates to certain 4,5-dihydroisoxazoles, to their use as estrogen receptor modulators, and to methods of their preparation.

### BACKGROUND OF THE INVENTION

The nuclear hormone receptor superfamily is a very important target for drug development. Members of this group include estrogen, androgen, progesterone, and glucocorticoid receptors, the activity of which can be controlled by ligand binding, as well as constitutive androstane and pregnane X receptors which have an essential role in drug metabolism. Nuclear hormone receptors regulate expression of their target genes, which control the essential metabolic reactions and differentiation processes of cells. Thus, nuclear receptor disorders are of great clinical importance.

Naturally occurring and synthetic estrogens have been widely utilized in the treatment of variety of disorders including cardiovascular disease, menopausal symptoms, dysmenorrhea, acne, prostatic cancer, hirsutism, osteoporosis and hot flashes. On the other hand, estrogen antagonists can be used in the treatment of diseases or conditions such as breast cancer, osteoporosis and anovulation. Because of this huge therapeutic value, there is a continuous need for artificial compounds which mimic estrogen-like behaviour in such a way that they have selective effects on different estrogen responsive tissues (selective estrogen receptor modulators i.e. SERMs). Also, it should be possible to utilize such compounds without the negative side effects of the estrogen replacement therapy. There are two subtypes of estrogen receptors: ERα and ERβ. Both forms bind to and are activated by their common natural ligand 3,17β-estradiol (E2), and none of the ER agonists or antagonists currently in clinical use are specific for either form. Because of the serious adverse effects of ER agonists and antagonists, great variation in ERα and β expression in diverse target tissues, and cell- and promoter-specific functions displayed by the ER subtypes, there are increasing efforts to explore new chemical scaffolds to develop both subtype-specific and tissue selective ligands.

Various methods for preparing isoxazoles and isoxazole derivatives have been disclosed for example in a) Bull. Chem. Soc. Jpn. 1984, 57, 2531-2534; b) Bull. Chem. Soc. Jpn. 1999, 72(10), 2277-2285; c) Org. Lett. 2000, 2(4), 539-541; and d) Tetrahedron 2000, 56, 1057-1064. Further methods are disclosed in publications e) Gazz. Chim. It. 1952, 82, 823-827; f) Kyushu Kyoritsu Daigaku Kenkyu Hokoku, Kogakubu 1987, 11, 1-8; g) Synthesis 1989, (1), 57-59; and h) Monatsh. Chem. 1901, 22, 750. However, the above mentioned publications do not suggest any pharmaceutical use for the disclosed compounds.

In conclusion, there is a need for small non-steroidal molecules which can act as agonists or antagonists for nuclear hormone receptors such as ERα and ERβ. We now describe a novel set of such compounds having estrogen activity in vitro and a further group of such compounds which are for the first time disclosed to have estrogen activity in vitro.

### DESCRIPTION OF THE INVENTION

This invention provides a novel compound of the formula (I)

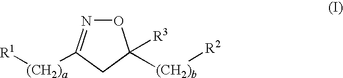

or a stereoisomer, pharmaceutically acceptable salt or a prodrug form thereof,

wherein

a is an integer from 0 to 3 and b is an integer from 0 to 2, provided that a and b are not simultaneously 0 or 1;

R¹ and R² are the same or different phenyl or naphthyl groups of the formula

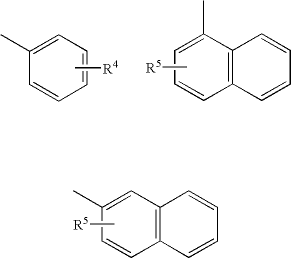

and are substituted with 0-5 R⁴ or R⁵;

R⁴ or R⁵ are the same or different and are selected from hydrogen, lower alkyl, lower alkoxyl, halogen, nitro, cyano, amino or hydroxyl;

R³ is selected from hydrogen, C₁₋₄alkyl, phenyl or benzyl;

provided that
when R³ is hydrogen, then R¹ and R² cannot simultaneously be an unsubstituted phenyl [a)-d)],
when a is 0, b is 1 and R³ is hydrogen, then R¹ cannot be an unsubstituted phenyl if R² is 4-hydroxy-3-methoxyphenyl,^{e} 3,4-dimethoxyphenyl,^{e} piperonyl,^{e} 1,4-dimethoxypiperonyl^{e} or 1-hydroxynaphthalen-2-yl,^{f} and then R¹ cannot be 4-methoxyphenyl if R² is 3,4-dimethoxyphenyl^{g} or piperonyl,^{g} and then R¹ cannot be 3,4,5-trimethoxyphenyl if R² is 3,4-dimethoxyphenyl^{g} or piperonyl^{g} [e)-g)], and
when a is 1, b is 0 and R³ is hydrogen, then R¹ cannot be unsubstituted phenyl if R² is 4-methylphenyl or 4-methoxyphenyl [h)], and
when a is 3, b is 0 and R³ is methyl, then R¹ cannot be unsubstituted phenyl if R² also is unsubstituted phenyl [b)].

In the compounds of formula (I), integer a is preferably 2 or 3, but it may also be 0 or 1. Integer b in the compounds of formula (I) is preferably 0 or 1, but it may also be 2. However, integers a and b are not both simultaneously 0 or 1. In the preferred compounds of formula (I), a is 2 or 3 and b is 1.

R¹ and R² may independently of each other be unsubstituted or ortho-, meta- or para-substituted by 0-5 substituent groups R⁴ or R⁵. Preferably groups R¹ and R² are independently of each other unsubstituted or substituted by one substituent R⁴ or R⁵.

In the preferred compounds of formula (I), substituents R⁴ and R⁵ are selected from the group consisting of hydrogen, lower alkyl, lower alkoxyl, halogen, nitro, cyano and hydroxyl. Alkyl or alkoxyl groups may be further substituted by the above mentioned groups.

Even more preferably substituents R⁴ and R⁵ are lower alkoxy, halogen or hydroxyl.

In the preferred compounds of formula (I), substituent R³ is hydrogen or a C₁₋₄ alkyl, especially methyl.

In the preferred compounds of formula (I), R¹ and R² are independently an unsubstituted phenyl or phenyl monosubstituted with alkoxy, halogen or hydroxyl.

In the context of the present application, the general terms used above and below preferably have the following meanings:

A compound having estrogen activity means a compound which acts as an agonist, antagonist, partial agonist or inverse agonist for nuclear hormone receptors such as ERα and ERβ.

A prodrug is a drug which is administered in an inactive or significantly less active form but once administered, it is metabolised in vivo into the active compound.

Alkyl is a saturated hydrocarbon radical containing 1-20, preferably 1-8 carbon atoms. It is for example methyl, ethyl, n-propyl, isopropyl, n-butyl, isobutyl, sec-butyl, tert-butyl, pentyl, hexyl, heptyl or octyl.

Lower alkyl contains 1-6, preferably 1-4 carbon atoms. It is for example ethyl, methyl, n-propyl, isopropyl, n-butyl, isobutyl, sec-butyl, tert-butyl, pentyl or hexyl, preferably methyl, ethyl, n-propyl or isopropyl.

In this description lower alkoxy contains 1-6, preferably 1-2 carbon atoms. It is for example methoxy, ethoxy, n-propoxy, isopropoxy, n-butoxy, isobutoxy, sec-butoxy, tert-butoxy, pentyloxy or ethyloxy, preferably methoxy.

Halogen is chlorine, bromine, fluorine or iodine, preferably fluorine or chlorine.

The compounds of the invention may contain one or more asymmetric centers and can thus occur as racemates and racemic mixtures, single enantiomers, diastereomeric mixtures and individual diastereomers. The present invention is meant to comprehend all such isomeric forms of the compounds of the invention.

The invention relates particularly to the compounds of formula (I)

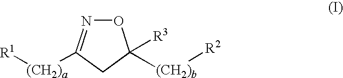

or stereoisomers, pharmaceutically acceptable salts or prodrug forms thereof,

wherein a is 2 or 3 and b is 0 or 1,

R¹ and R² are both phenyl groups which are independently substituted by one substituent selected from the group consisting of alkoxy, halogen or hydroxyl, and

R³ is hydrogen.

Preferred examples of the compounds of the invention are selected from the group consisting of 5-(4-fluorobenzyl)-3-phenethyl-4,5-dihydroisoxazole (66),

5-Benzyl-3-(4-hydroxy-phenyl)-4,5-dihydro-isoxazole (80),

3-(4-Hydroxy-benzyl)-5-(3-hydroxy-phenyl)-4,5-dihydro-isoxazole (83),

3-[2-(2-Hydroxy-phenyl)-ethyl]-5-methyl-5-phenyl-4,5-dihydro-isoxazole (85),

3-[2-(4-Hydroxy-phenyl)-ethyl]-5-methyl-5-phenyl-4,5-dihydro-isoxazole (87),

3-[3-(4-Hydroxy-phenyl)-propyl]-5-phenyl-4,5-dihydroisoxazole (88),

3-[3-(4-Hydroxy-phenyl)-propyl]-5-methyl-5-phenyl-4,5-dihydro-isoxazole (90),

5-Benzyl-3-[2-(2-hydroxy-phenyl)-ethyl]-4,5-dihydro-isoxazole (91),

5-Benzyl-3-[2-(4-hydroxy-phenyl)-ethyl]-4,5-dihydro-isoxazole (93),

5-(4-Hydroxy-benzyl)-3-phenethyl-4,5-dihydro-isoxazole (94),

5-(4-Hydroxy-benzyl)-3-[2-(4-hydroxy-phenyl)-ethyl]-4,5-dihydro-isoxazole (96),

5-(4-Fluoro-benzyl)-3-[2-(2-hydroxy-phenyl)-ethyl]-4,5-dihydro-isoxazole (97),

5-(4-Fluoro-benzyl)-3-[2-(4-hydroxy-phenyl)-ethyl]-4,5-dihydro-isoxazole (98),

5-Benzyl-3-[3-(4-hydroxy-phenyl)-propyl]-4,5-dihydro-isoxazole (99),

5-(4-Hydroxy-benzyl)-3-(3-phenyl-propyl)-4,5-dihydro-isoxazole (100),

3-[2-(4-Hydroxy-phenyl)-ethyl]-5-phenethyl-4,5-dihydro-isoxazole (106), and

stereoisomers, pharmaceutically acceptable salts and prodrug forms thereof.

The invention also relates to pharmaceutical compositions which contain a compound of the formula (I) or a stereoisomer, pharmaceutically acceptable salt or prodrug form thereof as active ingredient. These pharmaceutical compositions are for example those for enteral, such as in particular oral, those for parenteral administration, and those for local administration to warm-blooded animals, in particular to humans.

The pharmaceutical compositions according to the invention usually contain the pharmacologically active ingredient according to formula (I) together with known pharmaceutical excipients. The amount of the active ingredient in the pharmaceutical compositions according to the invention is, for example, from about 0.001% to 100% by weight, preferably from about 0.1% to about 50% by weight. The dose of the active ingredient can depend on various factors, such as the efficacy of the active ingredient, severity of the disease to be treated or its symptoms, administration procedure, sex, age, weight and/or individual condition of the subject in need of the treatment. In a normal case, for a human adult of about 75 kg in weight, one daily dose of about 1 mg to about 1000 mg, in particular from about 10 mg to about 500 mg, is to be estimated. This can be administered as a single dose or in several subdoses.

The invention also relates to the use of the compounds of the formula (I) or isomers, pharmaceutically acceptable salts or prodrug forms thereof for the preparation of pharmaceutical compositions for the treatment of disease states, disorders and conditions alleviated by compounds having estrogen activity. Among such conditions may be mentioned bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

The invention also provides the compounds of formula (I) or stereoisomers, pharmaceutically acceptable salts or prodrug forms thereof for use in a method for treating disease states, disorders or conditions alleviated by compounds having estrogen activity.

The invention also provides a method for the therapeutic or prophylactic treatment of disease states, disorders and conditions alleviated by compounds having estrogen activity, said method comprising administering an effective amount of a compound of formula (I) as defined above or a stereoisomer, pharmaceutically acceptable salt or prodrug form thereof to a subject in need of such treatment.

A still further object of the invention is a method for the therapeutic or prophylactic treatment of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate, said method comprising administering an effective amount of a compound of formula (I) as defined above or a stereoisomer, pharmaceutically acceptable salt or prodrug form thereof to a subject in need of such treatment.

The compounds of the formula (I) can be prepared as described below. The reagents used in the preparation of the compounds of this invention can be either commercially obtained or can be prepared by standard procedures described in the literature.

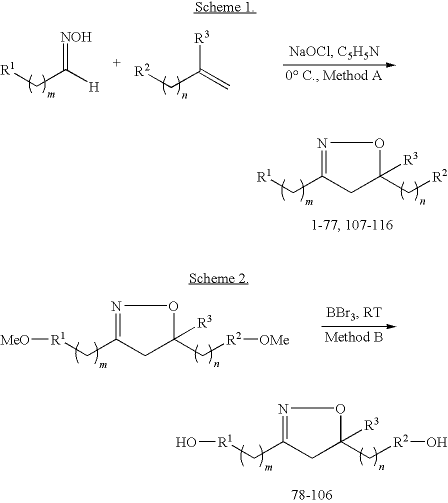

A process for preparing the compounds of formula (I) thus comprises
Scheme 1 (examples 1-77 and 107-116): nitrile oxide—olefin cycloaddition reaction of an aldoxime comprising R¹, wherein R¹ is as defined above, with an olefin comprising R² and R³, wherein R² and R³ are as defined above, in the presence of sodium hypochlorite and pyridine to afford the desired 4,5-dihydroisoxazoles; or
Scheme 2 (examples 78-106): demethylation reaction of the appropriate methoxy-substituted compounds in the presence of boron tribromide to afford the desired hydroxy-substituted products.

The invention also relates to the compounds of formula (I)

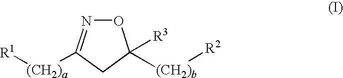

or stereoisomers, pharmaceutically acceptable salts or prodrug forms thereof,

wherein

a is an integer from 0 to 3 and b is an integer from 0 to 2, provided that a and b are not simultaneously 0 or 1;

R¹ and R² are the same or different phenyl or naphthyl groups of the formula

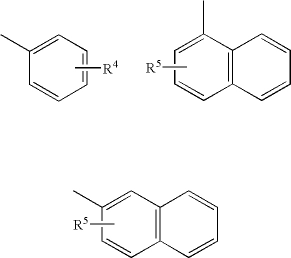

and are substituted with 0-5 R⁴ or R⁵;

R⁴ or R⁵ are the same or different and are selected from hydrogen, lower alkyl, lower alkoxyl, halogen, nitro, cyano, amino or hydroxyl;

R³ is selected from hydrogen, C₁₋₄alkyl, phenyl or benzyl, for use as pharmaceuticals, and for use in in a method for treating disease states, disorders or conditions alleviated by compounds having estrogen activity.

Further objects of the invention are
a pharmaceutical composition comprising a compound of the formula (I)

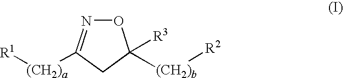

or a stereoisomer, pharmaceutically acceptable salt or a prodrug form thereof,

wherein

a is an integer from 0 to 3 and b is an integer from 0 to 2, provided that a and b are not simultaneously 0 or 1;

R¹ and R² are the same or different phenyl or naphthyl groups of the formula

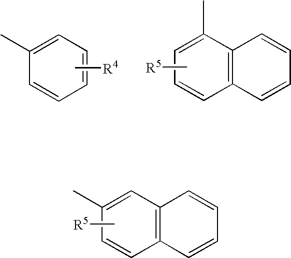

and are substituted with 0-5 R⁴ or R⁵;

R⁴ or R⁵ are the same or different and are selected from hydrogen, lower alkyl, lower alkoxyl, halogen, nitro, cyano, amino or hydroxyl;

R³ is selected from hydrogen, C₁₋₄alkyl, phenyl or benzyl, in association with a pharmaceutically acceptable carrier, and
the use of said compounds for the manufacture of medicaments for the treatment of disease states, disorders or conditions alleviated by compounds having estrogen activity.

The invention also provides a method for treating disease states, disorders or conditions alleviated by compounds having estrogen activity, said method comprising administering an effective amount of a compound of formula (I)

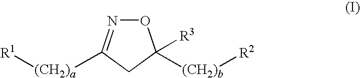

or stereoisomers, pharmaceutically acceptable salts or prodrug forms thereof,

wherein

a is an integer from 0 to 3 and b is an integer from 0 to 2, provided that a and b are not simultaneously 0 or 1;

R¹ and R² are the same or different phenyl or naphthyl groups of the formula

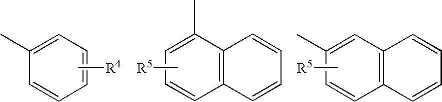

and are substituted with 0-5 R⁴ or R⁵;

R⁴ or R⁵ are the same or different and are selected from hydrogen, lower alkyl, lower alkoxyl, halogen, nitro, cyano, amino or hydroxyl;

R³ is selected from hydrogen, C₁₋₄alkyl, phenyl or benzyl, to a subject in need of such treatment.

The following examples further illustrate the invention described above.

### Example 1-77

### 5-Benzyl-3-(2-nitrophenyl)-4,5-dihydroisoxazole (1)

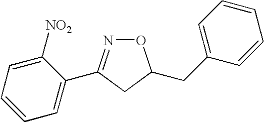

Method A: 2-Nitrobenzaldehyde oxime (2.14 g, 0.0129 mol) is dissolved into 100 ml of chloroform. Allylbenzene (2.31 g, 0.0194 mol) and pyridine (0.31 g, 0.0039 mol) are then added and the solution is cooled to 0° C. After that, 5% NaOCl solution (58 ml, 0.039 mol) is added dropwise to the vigorously stirred reaction mixture keeping the temperature of the solution at 0-5° C. for 1.5 h. Then the mixture is allowed to warm to the room temperature. The organic layer is separated and the water phase extracted with dichloromethane. The combined organic phase is then washed with 2 M HCl, saturated NaHCO₃ and water, dried with MgSO₄ and evaporated to dryness. The residue is purified by column cromatography using dichloromethane as an eluent.

Yield 78%, a pale yellow wax, ¹H NMR (CDCl₃): δ 8.05 (dd, 1H, J=8.1, 1.4), 7.66-7.55 (m, 2H), 7.43 (dd, 1H, J=7.6, 1.6), 7.35-7.23 (m, 5H), 5.08 (m, 1H), 3.22 (dd, 1H, J=16.7, 10.2), 3.18 (dd, 1H, J=13.7, 5.9), 3.01 (dd, 1H, J=13.7, 7.3), 2.99 (dd, J=16.7, 6.9); ¹³C NMR δ 155.2, 147.9, 136.6, (3 s), 133.5, 131.1, 130.5, 129.6, 128.7, 126.8 (6 d), 125.9 (s), 124.8, 82.7 (2 d), 41.3, 40.7 (2 t).

The following compounds included in the invention were prepared by Method A using appropriate starting materials:

### 5-(4-Methoxybenzyl)-3-(2-nitrophenyl)-4,5-dihydroisoxazole (2)

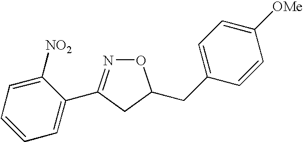

Yield 71%, a yellow oil, ¹H NMR (CDCl₃): δ 8.03 (dd, 1H, J=8.0, 1.2), 7.64 (ddd, 1H, J=7.5, 7.5, 1.3), 7.57 (ddd, 1H, J=8.1, 8.1, 1.6), 7.44 (dd, 1H, J=7.6, 1.5), 7.20 (m, 2H), 6.86 (m, 2H), 5.03 (m, 1H), 3.79 (s, 3H), 3.20 (dd, 1H, J=16.6, 10.2), 3.10 (dd, 1H, J=13.9, 5.9), 2.98 (dd, 1H, J=16.6, 7.0), 2.94 (dd, J=13.9, 7.2); ¹³C NMR δ 158.5, 155.2, 147.9 (3 s), 133.5, 131.1, 130.5 (3 d), 128.7, 125.9 (2 s), 124.7, 114.0, 82.9 (3 d), 55.3 (q), 41.2, 39.8 (2 t).

### 5-(3,4-Dimethoxybenzyl)-3-(2-nitrophenyl)-4,5-dihydroisoxazole (3)

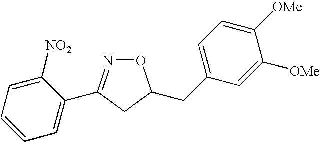

Yield 78%, a yellow oil, ¹H NMR (CDCl₃): δ 8.04 (dd, 1H, J=8.1, 1.2), 7.65 (ddd, 1H, J=7.5, 7.5, 1.3), 7.58 (ddd, 1H, J=8.0, 8.0, 1.6), 7.48 (dd, 1H, J=7.5, 1.5), 6.87-6.82 (m, 5H), 5.06 (m, 1H), 3.90, (s, 3H), 3.86 (s, 3H), 3.20 (dd, 1H, J=16.7, 10.2), 3.11 (dd, 1H, J=13.9, 6.0), 3.00 (dd, 1H, J=16.7, 6.8), 2.94 (dd, J=13.9, 7.2); ¹³C NMR δ 155.2, 149.0, 148.0, 147.9 (4 s), 133.5, 131.1, 130.6, 129.2 (4 d), 125.9 (s), 124.7, 121.4, 112.7, 111.3, 82.8 (5 d), 55.9, 55.9 (2 q), 41.2, 40.2 (2 t).

### 5-Benzyl-3-(4-nitrophenyl)-4,5-dihydro-isoxazole (4)

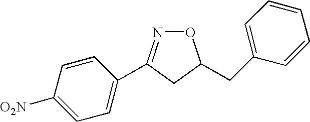

Yield 22%, a colorless wax, ¹NMR δ 8.23 (m, 2H), 7.78 (m, 2H), 7.34-7.30 (m, 2H), 7.28-7.23 (m, 3H), 5.09 (m, 1H), 3.34 (dd, 1H, J=16.6, 10.5), 3.18 (dd, 1H, J=14.0, 6.1), 3.08 (dd, 1H, J=16.6, 8.1), 2.95 (dd, J=14.0, 7.0); ¹³C NMR δ 155.0, 148.4, 136.4, 135.8, (4 s), 129.4, 128.7, 127.3, 127.0, 124.0, 83.0 (6 d), 40.9, 38.7 (2 t).

### 5-(4-Fluorobenzyl)-3-(4-nitrophenyl)-4,5-dihydroisoxazole (5)

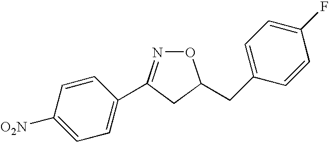

Yield 28%, a colourless wax; ¹NMR δ 8.24 (m, 2H), 7.79 (m, 2H), 7.24 (m, 2H), 7.01 (m, 2H), 5.06 (m, 1H), 3.37 (dd, 1H, J=16.6, 10.5), 3.11 (dd, 1H, J=14.2, 6.4), 3.06 (dd, 1H, J=16.6, 8.1), 2.96 (dd, 1H, J=14.2, 6.4); ¹³C NMR δ 161.9 (d), 154.9, 148.5, 135.7 (3 s), 132.0 (d), 131.0 (dd), 127.3, 124.0 (2 d), 115.6 (dd), 82.8 (d), 40.1, 38.7 (2 t).

### 5-Benzyl-3-(2-methoxyphenyl)-4,5-dihydroisoxazole (6)

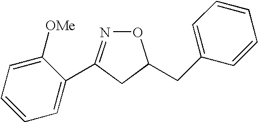

Yield 85%, mp=81.0-82.0° C., ¹H NMR (CDCl₃): δ 7.64 (dd, 1H, J=7.7, 1.7), 7.35-7.18 (m, 6H), 6.92 (ddd, 1H, J=7.6, 7.6, 1.0), 6.88 (dd, 1H, J=8.3, 0.9), 4.89 (m, 1H), 3.77 (s, 3H), 3.40 (dd, 1H, J=17.3, 10.1), 3.15 (dd, 1H, J=17.3, 7.5), 3.09 (dd, 1H, J=13.8, 6.2), 2.85 (dd, J=13.8, 7.0); ¹³C NMR δ 157.6, 156.2, 137.4 (3 s), 131.3, 129.5, 129.5, 128.6, 126.7, 120.8 (6 d), 119.1(s), 111.4, 81.8 (2 d), 55.4 (q), 42.0, 41.0 (2 t).

### 5-(4-Methoxybenzyl)-3-(2-methoxyphenyl)-4,5-dihydroisoxazole (7)

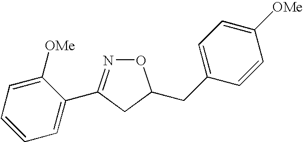

Yield 92%, a pale yellow oil, ¹H NMR (CDCl₃): δ 7.65 (dd, 1H, J=7.7, 1.7), 7.36 (ddd, 1H, J=8.4, 7.5, 1.8), 7.18 (m, 2H), 6.96 (ddd, 1H, J=7.6, 7.6, 1.0), 6.91 (d, 1H, J=8.4), 6.85 (m, 2H), 4.88 (m, 1H), 3.82 (s, 3H), 3.79 (s, 3H), 3.42 (dd, 1H, J=17.2, 10.1), 3.15 (dd, 1H, J=17.2, 7.5), 3.06 (dd, 1H, J=13.9, 6.1), 2.82 (dd, J=13.9, 7.1); ¹³C NMR δ 158.4, 157.5, 156.2 (3 s), 131.2, 130.4, 129.5 (3 d), 129.3 (s), 120.8 (d), 119.1 (s), 114.0, 111.3, 81.9 (3 d), 55.5, 55.3 (2 q), 42.0, 40.1 (2 t).

### 5-(3,4-Dimethoxybenzyl)-3-(2-methoxyphenyl)-4,5-dihydroisoxazole (8)

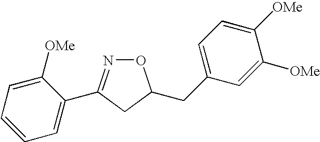

Yield 87%, a pale yellow oil, ¹H NMR (CDCl₃): δ 7.66 (dd, 1H, J=7.7, 1.7), 7.36 (ddd, 1H, J=8.4, 7.4, 1.8), 6.96 (ddd, 1H, J=7.6, 7.5, 1.0), 6.91 (d, 1H, J=8.4), 6.84-6.78 (m, 3H), 4.91 (m, 1H), 3.88, (s, 3H), 3.85 (s, 3H), 3.82 (s, 3H), 3.44 (dd, 1H, J=17.3, 10.2), 3.16 (dd, 1H, J=17.3, 7.6), 3.05 (dd, 1H, J=14.0, 6.2), 2.84 (dd, J=14.0, 6.7); ¹³C NMR δ 157.5, 156.2, 148.9, 147.8 (4 s), 131.2, 129.9, 121.4 (4 d), 120.8, 119.0 (2 s), 112.6, 111.3, 111.2, 81.4 (4 d), 55.9, 55.9, 55.5 (3 q), 42.0, 40.6 (2 t).

### 5-(4-Fluorobenzyl)-3-(2-methoxyphenyl)-4,5-dihydro-isoxazole (9)

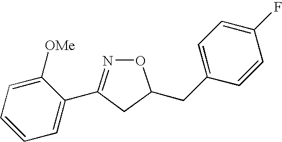

Yield 65%, a colourless wax; ¹H NMR δ 7.63 (dd, 1H, J=7.7, 1.7), 7.32 (m, 1H), 7.21 (m, 2H), 6.96 (m, 2H), 6.92 (dd, 1H, J=7.6, 7.5), 6.88 (d, 1H, J=8.4), 4.86 (m, 1H), 3.78 (s, 3H), 3.42 (dd, 1H, J=17.3, 10.2), 3.13 (dd, 1H, J=17.3, 7.5), 3.00 (dd, 1H, J=14.0, 6.6), 2.86 (dd, 1H, J=14.2, 6.2); ¹³C NMR δ 161.7 (d), 157.5, 156.1 (2 s), 133.1, 131.2 (2 d), 130.9 (dd), 129.3, 120.7 (2 d), 118.8 (s), 115.2 (dd), 111.5, 81.5 (2 d), 55.4 (s), 42.0, 40.1 (2 t).

### 5-Benzyl-3-(3-methoxyphenyl)-4,5-dihydro-isoxazole (10)

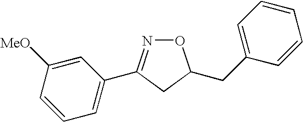

Yield 77%, a colourless wax, ¹H NMR δ 7.35-7.28 (m, 2H), 7.28-7.22 (m, 5H), 7.14 (d, 1H, J=7.7), 6.94 (dd, 1H, J=8.2, 2.4), 4.98 (m, 1H), 3.82 (s, 3H), 3.29 (dd, 1H, J=16.5, 10.2), 3.16 (dd, 1H, J=13.9, 6.1), 3.04 (dd, 1H, J=16.5, 7.8), 2.89 (dd, 1H, J=13.9, 7.3); ¹³C NMR δ 159.7, 156.4, 136.9, 131.0 (4 s), 129.7, 129.4, 128.6, 126.8, 119.3, 116.4, 111.2, 82.0 (8 d), 55.4 (q), 41.1, 39.4 (2 t).

### 5-(4-Fluorobenzyl)-3-(3-methoxyphenyl)-4,5-dihydro-isoxazole (11)

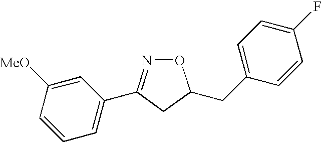

Yield 51%, a colourless wax; ¹H NMR δ 7.28 (dd, 1H, J=8.0, 7.9), 7.25-7.20 (m, 3H), 7.13 (d, 1H, J=7.7), 6.99 (m, 2H), 6.94 (dd, 1H, J=8.3, 2.1), 4.94 (m, 1H), 3.81 (s, 3H), 3.42 (dd, 1H, J=16.5, 10.3), 3.07 (dd, 1H, J=14.1, 6.5), 3.01 (dd, 1H, J=16.5, 7.8), 2.89 (dd, 1H, J=14.1, 6.4); ¹³C NMR δ 161.8 (d), 159.7, 156.4 (2 s), 132.6 (d), 130.9 (dd), 130.9 (s), 129.7, 119.3, 116.4 (3 d), 115.6 (dd), 111.3, 81.8 (2 d), 55.3 (s), 40.2, 39.4 (2 t).

### 5-Benzyl-3-(4-methoxyphenyl)-4,5-dihydroisoxazole (12)

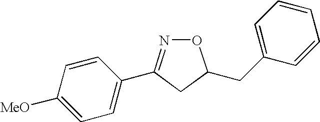

Yield 54%, mp=99.6-100.3° C., ¹H NMR (CDCl₃): δ 7.57 (m, 2H), 7.34-7.21 (m, 5H), 6.88 (m, 2H), 4.94 (m, 1H), 3.81 (s, 3H), 3.26 (dd, 1H, J=16.5, 10.2), 3.15 (dd, 1H, J=13.8, 6.1), 3.01 (dd, 1H, J=16.5, 7.8), 2.86 (dd, J=13.8, 7.3); ¹³C NMR δ 161.0, 156.0, 137.0, (3 s), 129.4, 128.6, 128.1, 126.7 (4 d), 122.2, (s), 114.1, 81.6 (2 d), 55.3 (q), 41.0, 39.6 (2 t).

### 5-(2-Methoxybenzyl)-3-(4-methoxyphenyl)-4,5-dihydroisoxazole (13)

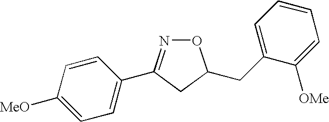

Yield 38%, a pale yellow wax; ¹H NMR δ 7.58 (m, 2H), 7.24-7.18 (m, 2H), 6.93-6.85 (m, 4H), 5.01 (m, 1H), 3.81 (s, 3H), 3.21 (dd, 1H, J=16.5, 10.1), 3.14 (dd, 1H, J=13.5, 6.0), 3.04 (dd, 1H, J=16.5, 7.3), 2.88 (dd, 1H, J=13.5, 7.7); ¹³C NMR δ 160.9, 157.6, 156.1 (3 s), 131.2, 128.1, 128.1 (3 d), 125.5, 122.6 (2 s), 120.6, 114.1, 110.4 (4 d), 80.4 (3 d), 55.3, 55.3 (2 q), 39.6, 35.7 (2 t).

### 5-(4-Methoxybenzyl)-3-(3-methoxyphenyl)-4,5-dihydroisoxazole (14)

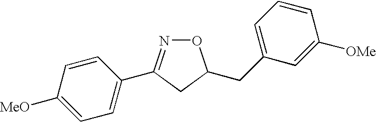

Yield 57%, a yellow wax, ¹H NMR δ 7.53 (m, 2H), 7.19 (dd, J=7.8, 7.8), 6.84 (m, 2H), 6.81-6.79 (m, 2H), 6.75 (dd, J=8.3, 2.0), 4.87 (m, 1H), 3.74 (s, 3H), 3.74 (s, 3H), 3.19 (dd, 1H, J=16.5, 10.2), 3.06 (dd, 1H, J=13.8, 6.3), 2.96 (dd, 1H, J=16.5, 8.0), 2.80 (dd, J=13.8, 7.0); ¹³C NMR δ 160.9, 159.7, 156.0, 138.7 (4 s), 129.5, 128.1 (2 d), 122.2 (s), 121.6, 115.1, 114.0, 111.9, 81.5 (5 d), 55.2, 55.1 (2 q), 41.0, 39.5 (2 t).

### 5-(4-Methoxybenzyl)-3-(4-methoxyphenyl)-4,5-dihydroisoxazole (15)

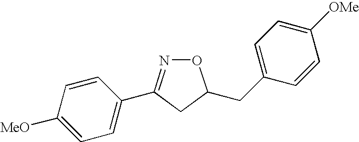

Yield 72%, mp=135.8-137.7° C., ¹H NMR (CDCl₃): δ 7.56 (m, 2H), 7.17 (m, 2H), 6.89 (m, 2H), 6.85 (m, 2H), 4.90 (m, 1H), 3.81 (s, 3H), 3.78 (s, 3H), 3.26 (dd, 1H, J=16.5, 10.1), 3.08 (dd, 1H, J=13.9, 5.9), 2.99 (dd, 1H, J=16.5, 7.8), 2.81 (dd, J=13.9, 7.2); ¹³C NMR δ 160.9, 158.4, 156.0, (3 s), 130.3 (d), 129.1 (s), 128.1 (d), 122.3 (s), 114.0, 81.8 (2 d), 55.3, 55.2 (2 q), 40.1, 39.5 (2 t).

### 5-(3,4-Dimethoxybenzyl)-3-(4-methoxyphenyl)-4,5-dihydroisoxazole (16)

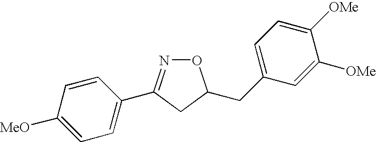

Yield 88%, mp=88.1-89.2° C., ¹H NMR (CDCl₃): δ 7.58 (m, 2H), 6.90 (m, 2H), 6.81 (m, 3H), 4.93 (m, 1H), 3.88, (s, 3H), 3.86 (s, 3H), 3.82 (s, 3H), 3.29 (dd, 1H, J=16.5, 10.2), 3.08 (dd, 1H, J=14.0, 6.2), 3.01 (dd, 1H, J=16.5, 7.9), 2.83 (dd, J=14.0, 6.8); ¹³C NMR δ 161.0, 156.1, 149.0, 147.9 (4 s), 129.7, 128.1 (2 d), 122.3, 121.4(2 s), 114.1, 112.6, 111.3, 81.7 (4 d), 55.9, 55.9, 55.3(3 q), 40.6, 39.6 (2 t).

### 5-{4-[2-(Dimethylamino)ethoxy]-benzyl}-3-(4-methoxyphenyl)-4,5-dihydro-isoxazole (17)

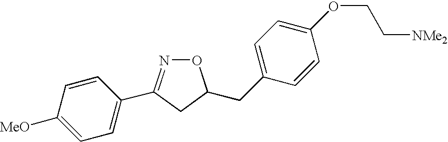

Yield 24%, a brown viscous oil, ¹H NMR δ 7.56 (m, 2H), 7.15 (m, 2H), 6.87 (m, 2H), 6.86 (m, 2H), 4.88 (m, 1H), 4.03 (t, 2H, J=5.7), 3.80 (s, 3H), 3.22 (dd, 2H, J=16.5, 6.5), 3.05 (dd, 2H, J=13.9, 6.0), 2.98 (dd, 2H, J=16.5, 7.8), 2.80 (dd, 2H, J=13.9, 7.0), 2.70 (t, 2H, J=5.7), 2.32 (s, 6H); ¹³C NMR δ 160.8, 157.6, 155.9 (3 s), 130.2 (d), 129.1, (s), 128.0 (d), 122.2 (s), 114.6, 114.0 (2 d), 81.6 (s), 65.9, 58.2 (2 t), 55.2 (q), 45.8, 40.0 (2 t), 39.4 (q).

### 5-(4-Fluorobenzyl)-3-(4-methoxyphenyl)-4,5-dihydroisoxazole (18)

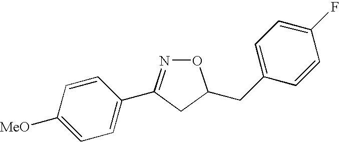

Yield 50%, mp=116.7-118.7° C.; ¹H NMR δ 7.56 (m, 2H), 7.22 (m, 2H), 6.99 (m, 2H), 6.88 (m, 2H), 4.90 (m, 1H), 3.81 (s, 3H), 3.29 (dd, 1H, J=16.4, 10.2), 3.07 (dd, 1H, J=14.1, 6.5), 2.98 (dd, 1H, J=16.4, 7.8), 2.87 (dd, 1H, J=14.1, 6.4); ¹³C NMR δ 161.8 (d), 161.0, 156.0 (2 s), 132.8 (d), 130.9 (dd), 128.1 (d), 122.2 (s), 115.4 (dd), 114.3, 81.4(2 d), 55.3 (q), 40.2, 39.7 (2 t).

### 3-(4-methoxyphenyl)-5-pentafluorobenzyl-4,5-dihydroisoxazole (19)

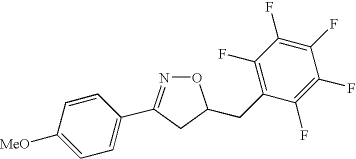

Yield 38%, mp=123.0-124.0° C., ¹H NMR δ 7.59 (m, 2H), 6.91 (m, 2H), 4.92 (m, 1H), 3.84 (s, 3H), 3.42 (dd, 1H, J=16.5, 10.1), 3.13 (dd, 1H, J=13.9, 7.4), 3.07 (dd, 1H, J=16.5, 6.7), 3.00 (dd, 1H, J=13.9, 5.9); ¹³C NMR δ 161.3, 156.0 (2 s), 145.5, 140.3, 137.6 (3 d), 128.3 (d), 121.8 (s), 114.4 (d), 110.6 (s), 78.8 (d), 55.4 (q), 40.2, 28.1 (2 t).

### 3-(4-Methoxy-phenyl)-5-(4-nitro-3-trifluoromethyl-benzyl)-4,5-dihydro-isoxazole (20)

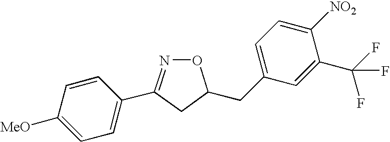

Yield 70%, a colourless wax; a colourless wax; ¹H NMR δ 7.82 (d, 1H, J=8.0), 7.77 (br s, 1H), 7.67 (d, 1H, J=8.0), 7.56 (m, 2H), 6.89 (m, 2H), 4.95 (m, 1H), 3.81 (s, 3H), 3.46 (dd, 1H, J=16.5, 10.0), 3.14 (dd, 1H, J=14.5, 8.0), 3.10 (dd, 1H, J=14.5, 4.5), 3.06 (dd, 1H, J=16.5, 7.5); ¹³C NMR δ 161.3, 156.4, 146.7 (3 s), 143.8 (d), 134.2 (s), 129.0 (dq), 128.3, 125.4 (2 d), 123.6, 122.1 (2 q), 121.7, 114.3 (2 d), 80.2 (d), 55.4 (q), 40.8, 40.3 (2 t).

### 4-[3-(4-Methoxy-phenyl)-4,5-dihydro-isoxazol-5-ylmethyl]-2-trifluoromethyl-benzonitrile (21)

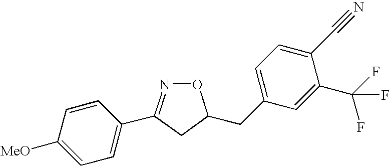

Yield 60%, a colourless wax; ¹H NMR δ 7.75 (d, 1H, J=7.9), 7.75 (br s, 1H), 7.65 (d, 1H, J=7.9), 7.56 (m, 2H), 6.89 (m, 2H), .4.95 (m, 1H), 3.82 (s, 3H), 3.46 (dd, 1H, J=16.5, 10.2), 3.13 (dd, 1H, J=14.4, 7.9), 3.09 (dd, 1H, J=14.4, 4.6), 3.05 (dd, 1H, J=16.5, 7.5); ¹³C NMR δ 161.6, 156.7 (2 s), 144.3, 135.2, 133.8 (3 d), 133.0 (q), 128.6 (d), 128.2 (dq), 122.8 (q), 122.0, 116.0, 114.6 (3 d), 108.5 (q), 80.5 (d), 55.7 (q), 41.5, 40.6 (2 t).

### 5-(1-Methoxy-2-naphthalenyl)methyl-3-(4-methoxyphenyl)-4,5-dihydroisoxazole (22)

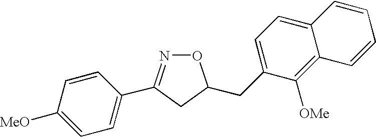

Yield 37%, a pale yellow wax; ¹H NMR δ 8.08 (d, 1H, J=8.3), 7.81 (d, 1H, J=8.0), 7.56 (m, 2H), 7.50 (m, 1H), 7.45 (m, 1H), 7.40 (d, 2H, J=8.4), 6.87 (m, 2H), 5.07 (m, 1H), 3.92 (s, 3H), 3.79 (s, 3H), 3.28 (dd, 1H, J=13.6, 6.1), 3.22 (dd, 1H, J=16.5, 10.1), 3.15-3.10 (m, 2H); ¹³C NMR δ 161.0, 156.3, 154.2, 134.3 (4 s), 128.6, 128.2 (2 d), 128.0 (s), 126.0, 125.8 (2 d), 125.5 (s), 124.3 (d), 122.4 (s), 122.1, 114.1, 81.2 (3 d), 62.0, 55.3 (2 q), 39.4, 35.1 (2 t).

### 5-[(3-Methoxy-naphthalen-2-yl)methyl]-3-(4-methoxyphenyl)-4,5-dihydroisoxazole (23)

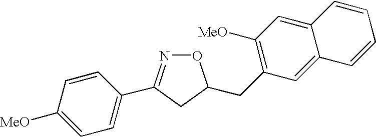

Yield 32%, a pale yellow wax, ¹H NMR δ 8.01 (d, 1H, J=8.6), 7.78 (d, 1H, J=7.6), 7.77 (d, 1H, J=9.0), 7.58 (m, 2H), 7.48 (m, 1H), 7.34 (m, 1H), 7.29 (d, 1H, J=9.0), 6.89 (m, 2H), 5.10 (m, 1H), 3.91 (s, 3H), 3.80 (s, 3H), 3.41 (d, 2H, J=7.2), 3.17 (dd, 1H, J=16.4, 6.8), 3.08 (dd, 1H, J=16.4, 10.0); ¹³C NMR δ 160.9, 156.5, 155.1, 133.5, 129.3 (5 s), 128.7, 128.6, 128.2, 126.8, 123.5 (5 d), 122.6 (s), 114.1, 113.3, 80.7 (3 d), 56.4, 55.3 (2 q), 39.3, 29.4 (2 t).

### 5-Benzyl-3-(3,4,5-trimethoxyphenyl)-4,5-dihydro-isoxazole (24)

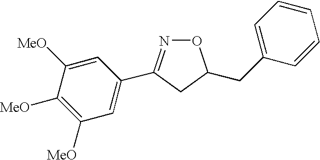

Yield 48%, a yellow wax, ¹H NMR δ 7.28 (m, 2H), 7.25-7.18 (m, 3H), 6.85 (s, 2H), 4.92 (m, 1H), 3,84 (s, 3H), 3.81 (s, 6H), 3.25 (dd, 1H, J=16.4, 10.2), 3.09 (dd, 1H, J=13.9, 6.3), 3.00 (dd, 1H, J=16.4, 8.0), 2.85 (dd, J=13.9, 6.9); ¹³C NMR δ 156.3, 153.2, 139.7, 137.0 (4 s), 129.2, 128.6, 126.7 (3 d), 125.2 (s), 103.7, 82.0 (2 d), 60.8, 56.1 (2 q), 40.9, 39.4 (2 t).

### 5-Benzyl-3-(2-fluorophenyl)-4,5-dihydro-isoxazole (25)

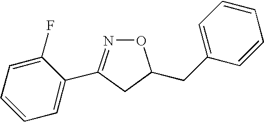

Yield 42%, a pale yellow wax, ¹H NMR δ 7.77 (m, 1H), 7.34-7.27 (m, 3H), 7.25-7.19 (m3H), 7.11 (dd, J=7.6, 7.5), 7.04 (dd, J=11.3, 8.4), 4.94 (m, 1H), 3.36 (ddd, 1H, J=17.2, 10.3, 2.4), 3.13 (ddd, 1H, J=17.2, 7.9, 2.3), 3.09 (dd, 1H, J=13.9, 6.2), 2.88 (dd, 1H, J=13.9, 6.8); ¹³C NMR δ 160.3 (d), 153.2, 136.9 (2 s), 131.6 (dd), 129.4 (d), 128.9 (dd), 128.6, 126.7 (2 d), 124.4 (dd), 117.8 (d), 116.3 (dd), 82.0 (d), 41.1 (td), 40.9 (t).

### 5-Benzyl-3-(3-fluorophenyl)-4,5-dihydro-isoxazole (26)

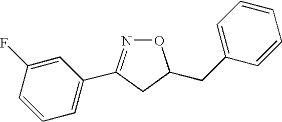

Yield 31%, a pale yellow wax, ¹H NMR δ 7.37-7.31 (m, 3H), 7.31-7.28 (m, 2H), 7.26-7.21 (m, 3H), 7.05 (m, 1H), 4.97 (m, 1H), 3.24 (dd, 1H, J=16.6, 10.3), 3.12 (dd, 1H, J=13.4, 6.2), 3.00 (dd, 1H, J=16.6, 7.9), 2.88 (dd, 1H, J=13.4, 7.1); ¹³C NMR δ 163.7, 155.6 (2d), 136.7 (s), 131.9 (d), 130.3 (dd), 129.4, 128.6, 126.8 (3 d), 122.4, 116.8, 113.4 (3 dd), 82.2 (d), 41.0, 39.2 (2 t).

### 5-Benzyl-3-(4-fluorophenyl)-4,5-dihydroisoxazole (27)

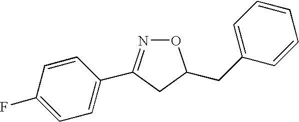

Yield 51%, a colourless wax; ¹H NMR δ 7.61 (m, 2H), 7.32 (m, 2H), 7.24 (m, 3H), 7.06 (m, 2H), 4.97 (m, 1H), 3.27 (dd, 1H, J=16.5, 10.3), 3.15 (dd, 1H, J=13.9, 6.1), 3.03 (dd, 1H, J=16.5, 7.8), 2.89 (dd, 1H, J=13.9, 7.2); ¹³C NMR δ 164.1 (d), 155.9, 137.2 (2 s), 129.8, 129.1 (2 d), 130.0 (dd), 127.2, 126.4 (2 d), 116.2 (dd), 82.4 (d), 41.4, 39.9 (2 t).

### 5-(4-Fluorobenzyl)-3-(4-fluorophenyl)-4,5-dihydroisoxazole (28)

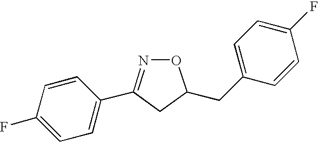

Yield 50%, a colourless wax; ¹H NMR δ 7.62 (m, 2H), 7.23 (m, 2H), 7.07 (m, 2H), 7.00 (m, 2H), 4.95 (m, 1H), 3.31 (dd, 1H, J=16.5, 10.3), 3.08 (dd, 1H, J=14.1, 6.4), 3.01 (dd, 1H, J=16.5, 7.9), 2.91 (dd, 1H, J=14.1, 6.5); ¹³C NMR δ 164.1, 162.3 (2 d), 155.9 (s), 132.9 (d), 131.3, 128.9 (2 dd), 126.3 (d), 116.2, 115.8 (2 dd), 82.2 (d), 40.6, 39.8 (2 t).

### 3-(4-Fluoro-phenyl)-5-(4-nitro-3-trifluoromethyl-benzyl)-4,5-dihydro-isoxazole (29)

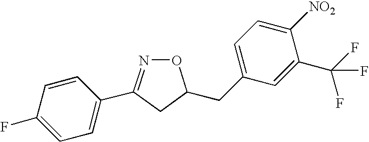

Yield 32%, a colourless wax; ¹H NMR δ 7.87 (d, 1H, J=8.0), 7.75 (br s, 1H), 7.68 (d, 1H, J=8.0), 7.63 (m, 2H), 7.10 (m, 2H), 5.02 (m, 1H), 3.49 (dd, 1H, J=16.5, 10.5), 3.18 (dd, 1H, J=14.5, 8.0), 3.11 (dd, 1H, J=14.5, 4.5), 3.05 (dd, 1H, J=16.5, 7.5); ¹³C NMR δ 164.0 (d), 155.6, 143.0, 134.0 (3 s), 128.8 (q), 128.6 (dd), 125.4 (d), 125.3 (d), 123.9, 122.0 (2 q), 118.3 (s), 116.0 (dd), 80.4 (d), 40.8, 40.0 (2 t).

### 4-[3-(4-Fluoro-phenyl)-4,5-dihydro-isoxazol-5-ylmethyl]-2-trifluoromethyl-benzonitrile (30)

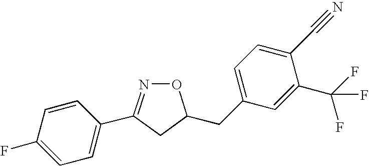

Yield 56%, a colourless wax; ¹H NMR δ 7.80 (d, 1H, J=7.9), 7.73 (br s, 1H), 7.64 (d, 1H, J=7.9), 7.63 (m, 2H), 7.10 (m, 2H), 5.01 (m, 1H), 3.48 (dd, 1H, J=16.5, 10.3), 3.16 (dd, 1H, J=14.4, 7.9), 3.10 (dd, 1H, J=14.5, 4.6), 3.04 (dd, 1H, J=16.5, 7.4); ¹³C NMR δ 163.9 (d), 155.6 (s), 143.3, 134.9, 133.3 (3 d), 133.0 (q), 128.6 (dd), 127.7 (dq), 125.3 (d), 123.3 (q), 116.0 (dd), 115.4 (d), 108.6 (q), 80.4 (d), 41.1, 40.0 (2 t).

### 5-Benzyl-3-(2-Chlorophenyl)-4,5-dihydroisoxazole (31)

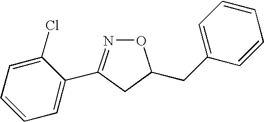

Yield 58%, a brown oil, ¹H NMR δ 7.44 (dd, 1H, J=7.6, 1.7), 7.31 (dd, 1H, J=8.0, 1.3), 7.27-7.14 (m, 7H), 4.93 (m, 1H), 3.36 (dd, 1H, J=17.0, 10.2), 3.13 (dd, 1H, J=17.0, 7.5), 3.03 (dd, 1H, J=13.9, 6.3), 2.88 (dd, 1H, J=13.9, 6.6); ¹³C NMR δ 156.2, 136.8, 132.5 (3 s), 130.6, 130.4, 130.3, 129.4 (4 d), 129.1 (s), 128.4, 126.8, 126.6 , 82.0 (3 d), 41.7, 40.6 (2 t).

### 5-Benzyl-3-(2-Chlorophenyl)-4,5-dihydroisoxazole (32)

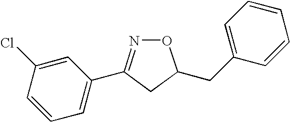

Yield 49%, a colourless viscous oil, ¹H NMR δ 7.58 (dd, 1H, J=1.7, 1.6), 7.47 (ddd, 1H, J=7.7, 1.7, 1.6), 7.31-7.20 (m, 7H), 4.96 (m, 1H), 3.21 (dd, 1H, J=16.6, 10.3), 3.11 (dd, 1H, J=13.9, 6.3), 2.97 (dd, 1H, J=16.6, 8.0), 2.87 (dd, 1H, J=13.9, 7.1); ¹³C NMR δ 155.4, 136.7, 134.6, 131.5 (4 s), 129.9, 129.8, 129.3, 128.6, 126.8, 126.5, 124.6, 82.2 (8 d), 40.9, 39.0 (2 t).

### 5-Benzyl-3-(4-Chlorophenyl)-4,5-dihydroisoxazole (33)

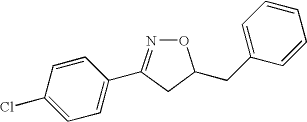

Yield 76%, mp=128.2-129.4° C., ¹H NMR (CDCl₃): δ 7.55 (m, 2H), 7.34 (m, 2H), 7.34-7.29 (m, 2H), 7.27-7.22 (m, 3H), 4.99 (m, 1H), 3.27 (dd, 1H, J=16.6, 10.3), 3.16 (dd, 1H, J=13.9, 6.1), 3.01 (dd, 1H, J=16.6, 7.9), 2.88 (dd, J=13.9, 7.2); ¹³C NMR δ 155.5, 136.7, 135.9, (3 s), 129.4, 128.9, 128.7 (3 d), 128.2 (s), 127.8, 126.8, 82.1 (2 d), 41.0, 39.2 (2 t).

### 3-(4-Chlorophenyl)-5-(4-ethylbenzyl)-4,5-dihydroisoxazole (34)

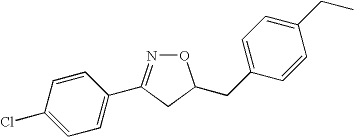

Yield 25%, mp=117.1-119.2° C.; ¹H NMR δ 7.56 (m, 2H), 7.35 (m, 2H), 7.16 (m, 2H), 7.15 (m, 2H), 4.97 (m, 1H), 3.26 (dd, 1H, J=16.6, 10.3), 3.13 (dd, 1H, J=13.9, 6.0), 3.02 (dd, 1H, J=16.6, 7.9), 2.86 (dd, 1H, J=13.9, 7.4); ¹³C NMR δ 155.5, 142.8, 135.9, 133.9 (4 s), 129.3, 128.9 (2 d), 128.3 (s), 128.2, 127.8, 82.3 (3 d), 40.6, 39.2, 28.5 (3 t), 15.6 (q).

### 3-(4-Chlorophenyl)-5-(4-isopropylbenzyl)-4,5-dihydro-isoxazole (35)

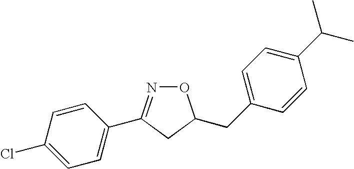

Yield 23%, a colourless wax; ¹NMR δ 7.56 (m, 2H), 7.35 (m, 2H), 7.16 (m, 2H), 7.15 (m, 2H), 4.97 (m, 1H), 3.26 (dd, 1H, J=16.6, 10.3), 3.13 (dd, 1H, J=13.9, 6.0), 3.02 (dd, 1H, J=16.6, 7.9), 2.86 (dd, 1H, J=13.9, 7.4); ¹³C NMR δ 155.5, 142.8, 135.9, 133.9 (4 s), 129.3, 128.9 (2 d), 128.3 (s), 128.2, 127.8, 82.3 (3 d), 40.6, 39.2, 28.5 (3 t), 15.6 (q).

### 3-(4-Chlorophenyl)-5-(2-methoxybenzyl)-4,5-dihydroisoxazole (36)

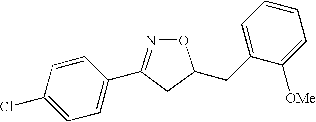

Yield 52%, mp=109.9-111.2° C.; ¹NMR δ 7.55 (m, 2H), 7.32 (m, 2H), 7.22 (ddd, 1H, J=7.7, 7.5, 1.6), 7.18 (d, 1H, J=7.5), 6.90 (ddd, 1H, J=7.7, 7.5, 0.9), 6.85 (d, 1H, J=7.7), 5.05 (m, 1H), 3.81 (s, 3H), 3.18 (dd, 1H, J=16.6, 10.3), 3.13 (dd, 1H, J=13.5, 6.0), 3.04 (dd, 1H, J=16.6, 7.5), 2.89 (dd, 1H, J=13.5, 7.7); ¹³C NMR δ 157.5, 155.6, 135.7 (3 s), 131.2, 128.9 (2 d), 128.5 (s), 128.1, 127.8 (2 d), 125.1 (s), 120.6, 110.4, 80.9 (3 d), 55.2 (q), 39.1, 36.7 (2 t).

### 3-(4-Chlorophenyl)-5-(3-methoxybenzyl)-4,5-dihydroisoxazole (37)

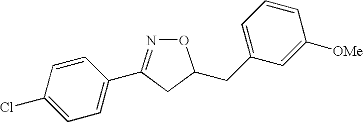

Yield 54%, a yellow wax, ¹H NMR δ 7.51 (m, 2H), 7.29 (m, 2H), 7.19 (dd, J=7.9, 7.9), 6.82-6.78 (m, 2H), 6.76 (dd, J=8.2, 2.1), 4.94 (m, 1H), 3.76 (s, 3H), 3.21 (dd, 1H, J=16.6, 10.3), 3.09 (dd, 1H, J=13.9, 6.2), 2.98 (dd, 1H, J=16.6, 8.1), 2.83 (dd, J=13.9, 7.1); ¹³C NMR δ 159.7, 155.5, 138.4, 136.7 (4 s), 129.6, 128.8 (2 d), 128.2 (s), 127.8, 121.6, 115.1, 112.0, 82.0 (5 d), 55.1 (q), 40.9, 39.2 (2 t).

### 3-(4-Chlorophenyl)-5-(4-methoxybenzyl)-4,5-dihydroisoxazole (38)

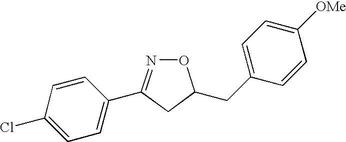

Yield 64%, mp=126.2-127.3° C, ¹H NMR (CDCl₃): δ 7.56 (m, 2H), 7.34 (m, 2H), 7.17 (m, 2H), 6.86 (m, 2H), 4.95 (m, 1H), 3.78 (s, 3H), 3.26 (dd, 1H, J=16.6, 10.3), 3.09 (dd, 1H, J=14.0, 6.0), 3.00 (dd, 1H, J=16.6, 7.9), 2.84 (dd, J=14.0, 7.1); ¹³C NMR δ 158.5, 155.5, 135.9, (3 s), 130.4, 128.9 (2 d), 128.7, 128.2 (2 s), 127.8, 114.1, 82.3 (2 d), 55.2 (q), 40.0, 39.1 (2 t).

### 3-(4-Chlorophenyl)-5-(3,4-dimethoxybenzyl)-4,5-dihydroisoxazole (39)

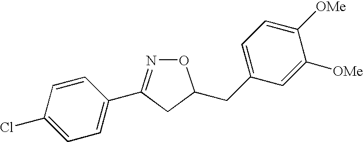

Yield 69%, mp=89.0-90.4° C., ¹H NMR (CDCl₃): δ 7.56 (m, 2H), 7.34 (m, 2H), 6.84-6.76 (m 3H), 4.99 (m, 1H), 3.88 (s, 3H), 3.86 (s, 3H), 3.28 (dd, 1H, J=16.6, 10.3), 3.09 (dd, 1H, J=14.0, 6.2), 3.01 (dd, 1H, J=16.6, 8.0), 2.86 (dd, 1H, J=14.0, 6.7); ¹³C NMR δ 155.6, 149.0, 147.9 135.9, 129.3 (5 s), 128.9 (d), 128.2 (s), 127.8, 121.3, 112.5, 111.3, 82.3 (5 d), 55.9, 55.9 (2 q), 40.5, 39.1 (2 t).

### 5-(4-Fluorobenzyl)-3-(4-chlorophenyl)-4,5-dihydro-isoxazole (40)

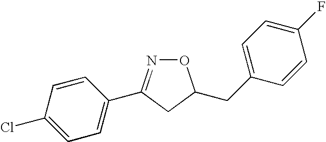

Yield 47%, a colourless wax; ¹H NMR δ 7.56 (m, 2H), 7.35 (m, 2H), 7.23 (m, 2H), 7.00 (m, 2H), 4.97 (m, 1H), 3.30 (dd, 1H, J=16.6, 10.3), 3.08 (dd, 1H, J=14.1, 6.4), 3.00 (dd, 1H, J=16.6, 7.9), 2.91 (dd, 1H, J=14.1, 6.5); ¹³C NMR δ 161.9 (d), 155.5, 136.0 (2 s), 132.4 (d), 130.9 (dd), 129.0 (d), 128.1 (s), 127.8 (d), 115.5 (dd), 82.0 (d), 40.2, 39.2 (2 t).

### 3-(4-Chlorophenyl)-5-naphthalen-1-ylmethyl-4,5-dihydro-isoxazole (41)

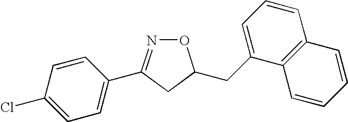

Yield 23%, mp=127.9-130.9° C.; ¹H NMR δ 8.08 (d, 1H, J=8.5), 7.89 (d, 1H, J=7.9), 7.79 (d, 1H, J=8.1), 7.58 (m, 2H), 7.53 (m, 1H), 7.51 (m, 1H), 7.44 (dd, 1H, J=8.0, 7.1), 7.39 (d, 1H, J=6.8), 7.36 (m, 2H), 5.21 (m, 1H), 3.71 (dd, 1H, J=14.1, 5.8), 3.28-3.21 (m, 2H), 3.11 (dd, 1H, J=16.6, 7.6); ¹³C NMR δ 155.6, 136.0, 134.0, 132.9, 132.0 (5 s), 129.0, 129.0 (2 d), 128.2 (s), 127.9, 127.8, 127.5, 126.3, 125.8, 125.5, 123.5, 81.3 (8 d), 39.5, 38.1 (2 t).

### 5-Benzyl-3-(2,6-dichlorophenyl)-4,5-dihydroisoxazole (42)

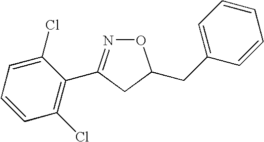

Yield 78%, mp=90.0-90.5° C., ¹H NMR (CDCl₃): δ 7.36-7.21 (m, 8H), 5.08 (m, 1H), 3.27 (dd, 1H, J=17.1, 10.3), 3.21 (dd, 1H, J=14.1, 5.9), 3.00 (dd, 1H, J=17.1, 7.0), 2.85 (dd, J=13.8, 7.5); ¹³C NMR δ 153.8, 136.7, 135.0 (3 s), 130.9, 129.5 (2 d), 129.0 (s), 128.7, 128.1, 126.8, 82.1 (4 d), 41.6, 40.8 (2 t).

### 3-(2,6-Dichlorophenyl)-5-(4-methoxybenzyl)-4,5-dihydroisoxazole (43)

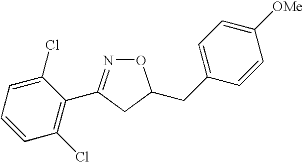

Yield 75%, mp=90.1-90.6° C., ¹H NMR (CDCl₃): δ 7.37-7.33 (m, 2H), 7.30-7.24 (m, 1H), 7.20 (m, 2H), 6.86 (m, 2H), 5.04 (m, 1H), 3.79 (s, 3H), 3.26 (dd, 1H, J=17.0, 10.3), 3.14 (dd, 1H, J=13.9, 5.9), 2.99 (dd, 1H, J=17.0, 7.0), 2.93 (dd, J=13.9, 7.4); ¹³C NMR δ 158.5, 153.8, 135.0 (3 s), 130.9, 130.5 (2 d), 129.1, 128.7 (2 s), 128.1, 114.1, 82.3 (3 d), 55.3 (q), 41.6, 39.9 (2 t).

### 3-(2,6-Dichlorophenyl)-5-(3,4-dimethoxybenzyl)-4,5-dihydroisoxazole (44)

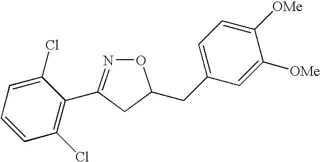

Yield 86%, a pale yellow oil, ¹H NMR (CDCl₃): δ 7.35 (d, 1H, J=9.0), 7.35 (d, 1H, J=7.0), 7.27 (dd, 1H, J=9.2, 7.0), 6.86-6.79 (m, 3H), 5.07 (m, 1H), 3.88 (s, 3H), 3.86 (s, 3H), 3.28 (dd, 1H, J=17.0, 10.4), 3.14 (dd, 1H, J=14.0, 6.0), 3.01 (dd, 1H, J=17.0, 7.1), 2.95 (dd, J=14.0, 7.0); ¹³C NMR δ 153.9, 149.0, 147.9, 135.0 (4 s), 131.0 (d), 129.3, 129.0 (2 s), 128.1, 121.5, 112.7, 111.3, 82.2 (5 d), 55.9, 55.9 (2 q), 41.6, 40.3 (2 t).

### 4-(5-Benzyl-4,5-dihydro-isoxazol-3-yl)-benzonitrile (45)

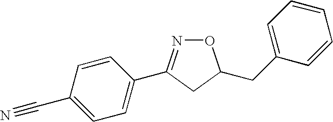

Yield 18%, a colorless wax ¹H NMR δ 7.72 (m, 2H), 7.66 (m, 2H), 7.34-7.30 (m, 2H), 7.28-7.24 (m, 3H), 5.07 (m, 1H), 3.30 (dd, 1H, J=16.6, 10.4), 3.17 (dd, 1H, J=14.0, 6.1), 3.04 (dd, 1H, J=16.6, 7.9), 2.93 (dd, J=14.0, 7.1); ¹³C NMR δ 155.2, 136.4, 134.0, (3 s), 132.4, 129.4, 128.7 (3d), 127.8 (s), 127.0, 127.0 (2 d), 118.3 (s), 82.8 (d), 40.9, 38.7 (2 t).

### 4-[5-(4-Fluorobenzyl)]-4,5-dihydro-isoxazol-3-yl)-benzo-nitrile (46)

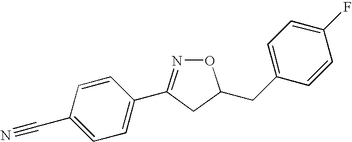

Yield 23%, a colourless wax; ¹H NMR δ 7.72 (m, 2H), 7.67 (m, 2H), 7.23 (m, 2H), 7.01 (m, 2H), 5.04 (m, 1H), 3.33 (dd, 1H, J=16.6, 10.5), 3.09 (dd, 1H, J=14.1, 6.5), 3.03 (dd, 1H, J=16.6, 8.0), 2.94 (dd, 1H, J=14.1, 6.3); ¹³C NMR δ 161.9 (d), 155.2, 133.9 (2 s), 132.5, 132.1 (2 d), 130.9 (dd), 127.0, 118.3 (2 d), 115.5 (dd), 82.6 (d), 40.1, 38.7 (2 t).

### 5-(4-Nitro-3-trifluoromethyl-benzyl)-3-phenyl-4,5-dihydro-isoxazole (47)

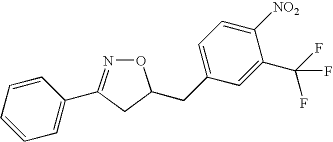

Yield 56%, a colourless wax; ¹H NMR δ 7.82 (d, 1H, J=8.5), 7.76 (br s, 1H), 7.67 (d, 1H, J=8.5), 7.63 (m, 2H), 7.42-7.36 (m, 3H), 4.99 (m, 1H), 3.49 (dd, 1H, J=16.5, 10.5), 3.15 (dd, 1H, J=14.0, 8.0), 3.10 (dd, 1H, J=14.0, 4.5), 3.08 (dd, 1H, J=16.5, 7.5); ¹³C NMR δ 156.7, 149.7, 143.5, 134.1 (4 s), 130.4 (d), 129.1 (s), 128.9 (q), 128.8, 126.7, 125.4 (3 d), 123.7, 122.2 (2 q), 80.4 (d), 40.8, 40.0 (2 t).

### 4-(3-Phenyl-4,5-dihydro-isoxazol-5-ylmethyl)-2-trifluoromethyl-benzonitrile (48)

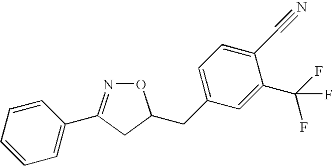

Yield 57%, a colourless wax; ¹H NMR δ 7.79 (d, 1H, J=7.9), 7.73 (br s, 1H), 7.64 (d, 1H, J=7.9), 7.63 (m, 2H), 7.45-7.38 (m, 3H), 5.01 (m, 1H), 3.50 (dd, 1H, J=17.0, 10.5), 3.16 (dd, 1H, J=14.5, 8.0), 3.10 (dd, 1H, J=14.5, 4.5), 3.07 (dd, 1H, J=17.0, 7.5); ¹³C NMR δ 156.7 (s), 143.8, 134.8, 133.5 (3 d), 132.6 (q), 130.4 (d), 129.1 (s), 128.8 (d), 127.8 (dq), 122.4 (q), 115.6 (d), 108.2 (q), 80.4 (d), 41.1, 40.0 (2 t).

### 5-benzyl-3-naphthalen-1-yl-4,5-dihydro-isoxazole (49)

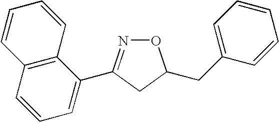

Yield 75%, a pale brown viscous oil, ¹H NMR δ 8.92 (d, 1H, J=8.6), 7.77-7.72 (m, 2H), 7.50 (ddd, 1H, J=7.6, 7.6, 1.3), 7.43 (ddd, 1H, J=7.6, 7.6, 1.1), 7.33-7.28 (m, 2H), 7.26 (m, 2H), 7.22-7.17 (m, 3H), 4.87 (m, 1H), 3.32 (dd, 1H, J=16.4, 9.2), 3.10-3.04 (m, 2H), 2.87 (dd, 1H, J=13.9, 6.7); ¹³C NMR δ 157.0, 136.9, 133.8 (3 s), 130.5 (d), 130.5 (s), 129.4, 128.6, 128.5, 127.5, 127.3, 127.0, 126.6 (7 d), 126.6 (s), 126.2, 124.7, 80.5 (3 d), 42.6, 40.8 (2 t).

### 5-benzyl-3-naphthalen-2-yl-4,5-dihydro-isoxazole (50)

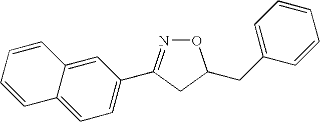

Yield 56%, a pale brown wax, ¹H NMR δ 7.92 (dd, 1H, J=8.7, 1.4), 7.78-7.72 (m, 4H), 7.49-7.40 (m, 2H), 7.29 (m, 2H), 7.25-7.20 (m, 3H), 4.96 (m, 1H), 3.29 (dd, 1H, J=16.4, 10.2), 3.13 (dd, 1H, J=13.9, 10.2), 3.08 (dd, 1H, J=16.4, 8.0), 2.87 (dd, 1H, J=13.9, 7.2); ¹³C NMR δ 156.5, 136.9, 133.9, 132.9 (4 s), 129.4, 128.6, 128.4, 128.3, 127.8 (5 d), 127.3 (s), 127.0, 126.7, 126.7, 126.6, 113.4, 82.0 (6 d), 41.0, 39.2 (2 t).

### 5-Benzyl-3-(2-methoxyphenyl)-5-methyl-4,5-dihydro-isoxazole (51)

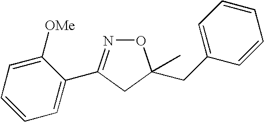

Yield 45%, a brown viscous, ¹H NMR δ 7.58 (dd, 1H, J=7.7, 1.8), 7.31 (m, 1H), 7.28-7.25 (m, 4H), 7.20 (m, 1H), 6.91 (ddd, 1H, J=7.6, 7.6, 1.0), 6.87 (d, 1H, J=8.4), 3.78 (s, 3H), 3.33 (d, 1H, J=17.2), 3.09 (d, 1H, J=17.2), 2.97 (s, 2H), 1.42 (s, 3H); ¹³C NMR δ 157.4, 156.2, 137.0 (3 s), 130.9, 130.4, 129.2, 128.1, 126.5, 120.7 (6 d), 119.4 (s), 111.3, 87.0 (2 d), 55.4 (q), 47.3, 45.7 (2 t), 25.4 (q).

### 5-Benzyl-3-(3-methoxyphenyl)-5-methyl-4,5-dihydro-isoxazole (52)

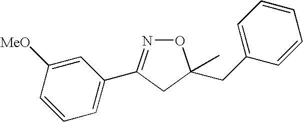

Yield 56%, a brown viscous oil, ¹H NMR δ 7.28-7.19 (m, 7H), 7.09 (d, 1H, J=7.7), 6.90 (dd, 1H, J=8.2, 2.1), 3.78 (s, 3H), 3.18 (d, 1H, J=16.5), 2.99 (s, 2H), 2.91 (d, 1H, J=16.5), 1.43 (s, 3H); ¹³C NMR δ 159.6, 156.4, 136.7, 131.4 (4 s), 130.3, 129.6, 128.2, 126.7, 119.1, 116.1, 111.0, 87.5 (8 d), 55.3 (q), 45.6, 44.5 (2 t), 25.8 (q).

### 5-Benzyl-3-(4-methoxyphenyl)-5-methyl-4,5-dihydro-isoxazole (53)

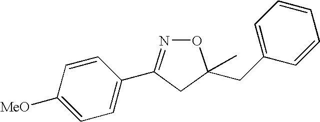

Yield 37%, mp=97.8-98.6° C.; ¹H NMR δ 7.53 (m, 2H), 7.29-7.27 (m, 4H), 7.22 (m, 1H), 6.88 (m, 2H), 3.80 (s, 3H), 3.20 (d, 1H, J=16.4), 2.94 (s, 2H), 2.92 (d, 1H, J=16.4), 1.43 (s, 3H); ¹³C NMR δ 160.6, 156.0, 136.9 (3 s), 130.4, 128.2, 127.9, 126.7 (4 d), 122.7 (s), 114.0, 87.0 (2 d), 55.3 (q), 45.7, 44.8 (2 t), 25.7 (q).

### 5-Benzyl-3-(2-fluorophenyl)-5-methyl-4,5-dihydro-isoxazole (54)

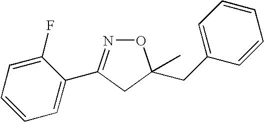

Yield 42%, a pale yellow wax, ¹H NMR δ 7.74 (ddd, J=7.7, 7.7, 1.7), 7.33 (m, 1H), 7.29-7.27 (m, 4H), 7.23 (m, 1H), 7.12 (ddd, J=7.6, 7.6, 1.1), 7.04 (ddd, J=11.3, 8.4, 0.9), 3.32 (dd, 1H, J=17.2, 2.4), 3.07 (dd, 1H, J=17.2, 2.6), 3.01 (d, 1H, J=14.3), 3.00 (d, 1H, J=14.3), 1.45 (s, 3H); ¹³C NMR δ 160.2, 153.2 (2 d), 136.7 (s), 131.4 (dd), 130.4 (d), 128.8 (dd), 128.2, 126.7 (2 d), 124.3 (dd), 118.2 (d), 116.3 (dd), 87.6 (d), 46.4 (td), 45.7 (t), 25.7 (q).

### 5-Benzyl-3-(4-chlorophenyl)-5-methyl-4,5-dihydro-isoxazole (55)

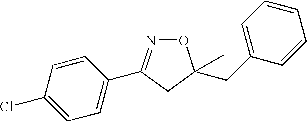

Yield 35%, a pale brown viscous oil, ¹H NMR δ 7.50 (m, 2H), 7.32 (m, 2H), 7.29-7.26 (m, 4H), 7.23 (m, 1H), 3.18 (d, 1H, J=16.5), 3.01 (d, 1H, J=13.9), 3.00 (d, 1H, J=13.9), 2.91 (d, 1H, J=16.5), 1.45 (s, 3H); ¹³C NMR δ 155.4, 136.6, 135.7 (3 s), 130.3, 128.8 (2 d), 128.6 (s), 128.3, 127.6, 126.8, 87.8 (4 d), 45.7, 44.3 (2 t), 25.9 (q).

### 4-(5-Benzyl-5-methyl-4,5-dihydro-isoxazol-3-yl)-benzonitrile (56)

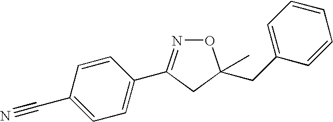

Yield 35%, a pale yellow wax, ¹H NMR δ 7.67 (m, 2H), 7.63 (m, 2H), 7.29-7.25 (m, 4 OH), 7.22 (m, 1H), 3.20 (d, 1H, J=16.5), 3.05 (d, 1H, J=13.9), 3.00 (d, 1H, J=13.9), 2.94 (d, 1H, J=16.5), 1.48 (s, 3H); ¹³C NMR δ 155.0, 136.3, 134.4 (3 s), 132.4, 130.3, 128.3 (3 d), 127.8 (s), 126.9, 126.8 (2 d), 118.4 (s), 88.8 (d), 45.7, 43.7 (2 t), 26.0 (q).

### 5-Benzyl-3-naphthalen-2-yl-5-methyl-4,5-dihydro-isoxazole (57)

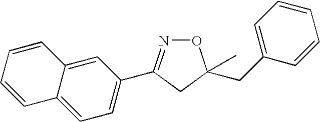

Yield 40%, mp=144.8-146.3° C.; ¹H NMR δ 7.92 (dd, 1H, J=8.7, 1.5), 7.82-7.78 (m, 4H), 7.49 (m, 2H), 7.38-7.27 (m, 4H), 7.22 (m, 1H), 3.35 (d, 1H, J=16.3), 3.07 (d, 1H, J=16.3), 3.06 (s, 2H), 1.49 (s, 3H); ¹³C NMR δ 156.6, 136.8, 133.9, 133.0 (4 s), 130.4, 128.4, 128.3, 128.3, 127.8 (5 d), 127.8 (s), 126.9, 126.8, 126.6, 126.5, 123.4, 87.7 (6 d), 45.8, 44.4 (2 t), 25.9 (q).

### 3-(2-Methoxy-phenyl)-5-phenethyl-4,5-dihydro-isoxazole (58)

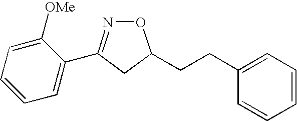

Yield 60%, a yellow wax; ¹H NMR δ 7.71 (dd, 1H, J=7.7, 1.7), 7.36 (m, 1H), 7.28 (m, 2H), 7.22 (m, 2H), 7.19 (m, 1H), 6.96 (dd, 1H, J=7.5, 7.5), 6.92 (d, 1H, J=8.4), 4.67 (m, 1H), 3.83 (s, 3H), 3.47 (dd, 1H, J=17.2, 10.2), 3.10 (dd, 1H, J=17.2, 8.1), 2.83 (ddd, 1H, J=14.1, 9.8, 5.5), 2.76 (ddd, 1H, J=14.1, 9.5, 6.8), 2.09 (m, 1H), 1.91 (m, 1H); ¹³C NMR δ 157.6, 156.2, 141.4, (3 s), 131.1, 129.4, 128.5, 128.4, 125.9, 120.8 (6 d), 119.1 (s), 111.4, 80.3 (2 d), 55.5 (q), 42.7, 37.0, 31.9 (3 t).

### 3-(3-Methoxy-phenyl)-5-phenethyl-4,5-dihydro-isoxazole (59)

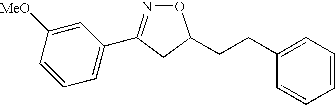

Yield 53%, a colourless viscous oil; ¹NMR δ 7.31-7.25 (m, 4H), 7.24-7.16 (m, 3H), 7.16 (m, 1H), 6.94 (d, 1H, J=8.8,), 4.72 (m, 1H), 3.35 (dd, 1H, J=16.5, 10.4), 2.95 (dd, 1H, J=16.5, 8.0), 2.82 (ddd, 1H, J=14.1, 9.3, 5.6), 2.77 (ddd, 1H, J=14.1, 9.3, 6.9), 2.10 (m, 1H), 1.92 (m, 1H); ¹³C NMR δ 159.7, 156.4, 141.1, 131.1 (4 s), 129.7, 128.5, 128.5, 126.1, 119.3, 116.3, 111.2, 80.5 (8 d), 55.3 (q), 40.0, 37.1, 31.8, 30.9 (4 t).

### 3-(4-Methoxy-phenyl)-5-phenethyl-4,5-dihydro-isoxazole (60)

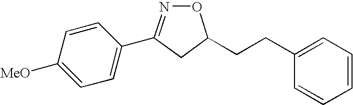

Yield 41%, mp=69.7-71.9° C.; ¹H NMR δ 7.57 (m, 2H), 7.26 (m, 2H), 7.19 (m, 2H), 7.17 (m, 1H), 6.87 (m, 2H), 4.64 (m, 1H), 3.77 (s, 3H), 3.28 (dd, 1H, J=16.4, 10.3), 2.89 (dd, 1H, J=16.4, 8.1), 2.80 (ddd, 1H, J=14.1, 9.8, 5.6), 2.73 (ddd, 1H, J=14.1, 9.4, 6.9), 2.06 (m, 1H), 1.89 (m, 1H); ¹³C NMR δ 160.9, 156.0, 141.2, (3 s), 128.4, 128.4, 128.1, 127.0 (4 d), 122.3 (s), 114.0, 80.1 (2 d), 55.3 (q), 40.0, 37.0, 31.9 (3 t).

### 3-(3-Fluoro-phenyl)-5-phenethyl-4,5-dihydro-isoxazole (61)

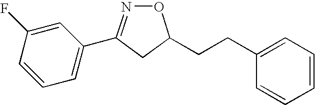

Yield 40%, a colourless viscous oil; ¹NMR δ 7.41-7.32 (m, 3H), 7.29 (m, 2H), 7.24-7.18 (m, 3H), 7.08 (m, 1H), 4.73 (m, 1H), 3.34 (dd, 1H, J=16.5, 10.5), 2.94 (dd, 1H, J=16.5, 8.1), 2.83 (ddd, 1H, J=14.1, 9.6, 5.7), 2.77 (ddd, 1H, J=14.1, 9.2, 7.0), 2.10 (m, 1H), 1.95 (m, 1H); ¹³C NMR δ 162.8, 155.6 (2 d), 141.0 (s), 132.0 (d), 130.3 (dd), 128.5, 128.5 (2 d), 126.1 (d), 122.3, 116.9, 113.4 (3 dd), 80.8 (d), 39.8, 37.1, 31.8 (3 t).

### 3-(4-Fluoro-phenyl)-5-phenethyl-4,5-dihydro-isoxazole (62)

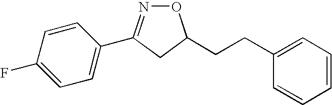

Yield 60%, mp=69.8-71.2° C.; ¹H NMR δ 7.63 (m, 2H), 7.28 (m, 2H), 7.23-7.16 (m, 3H), 7.06 (m, 2H), 4.70 (m, 1H), 3.32 (dd, 1H, J=16.4, 10.4), 2.93 (dd, 1H, J=16.4, 8.1), 2.83 (ddd, 1H, J=14.1, 9.6, 5.7), 2.76 (ddd, 1H, J=14.1, 9.3, 6.8), 2.10 (m, 1H), 1.92 (m, 1H); ¹³C NMR δ 163.7 (d), 155.5, 141.1 (s), 128.5 (d), 128.5 (dd), 128.5, 128.5, 126.1 (3 d), 115.8 (dd), 80.6 (d), 40.0, 37.0, 31.8 (3 t).

### 3-(4-Chloro-phenyl)-5-phenethyl-4,5-dihydro-isoxazole (63)

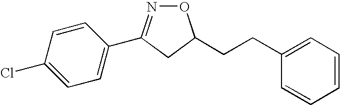

Yield 59%, mp=74.8-75.7° C.; ¹H NMR δ 7.58 (m, 2H), 7.36 (m, 2H), 7.29 (m, 2 H), 7.22 (m, 2H), 7.20 (m, 1H), 4.76 (m, 1H), 3.34 (dd, 1H, J=16.5, 10.5), 2.94 (dd, 1H, J=16.5, 8.1), 2.84 (ddd, 1H, J=15.0, 9.3, 5.5), 2.77 (ddd, 1H, J=15.0, 9.2, 6.9), 2.10 (m, 1H), 1.93 (m, 1H); ¹³C NMR δ 155.5, 141.0, 135.9 (3 s), 129.0, 128.5, 128.5 (3 d), 128.3 (s), 127.8, 126.1, 80.7 (3 d), 39.8, 37.1, 31.8 (3 t).

### 5-(2-Methoxybenzyl)-3-phenethyl-4,5-dihydroisoxazole (64)

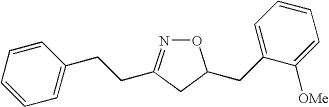

Yield 73%, a colourless viscous oil; ¹H NMR (CDCl₃): δ 7.37-7.31 (m, 2H), 7.29-7.23 (m, 4H), 7.17 (dd, 1H, J=7.4, 1.6), 6.95 (dt, 1H, J=7.4, 1.1), 6.90 (d, 1H, J=8.2), 4.89 (m, 1H), 3.85 (s, 2H), 3.08 (dd, 1H, J=13.4, 5.9), 2.91 (m, 1H), 2.88 (dd, 1H, J=17.0, 10.0), 2.80 (dd, 1H, J=13.4, 7.6), 2.70-2.64 (m, 2H).

### 5-(4-Methoxybenzyl)-3-phenethyl-4,5-dihydroisoxazole (65)

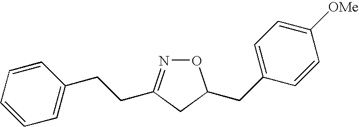

Yield 44%, a colourless wax; ¹H NMR δ 7.26 (m, 2H), 7.21-7.14 (m, 3H), 7.09 (m, 2H), 6.82 (m, 2H), 4.70 (m, 1H), 3.75 (s, 3H), 2.89 (dd, 1H, J=14.0, 6.0), 2.84-2.77 (m, 3H), 2.68 (dd, 1H, J=13.8, 6.8), 2.61-2.51 (m, 3H); ¹³C NMR δ 158.4, 158.1, 140.5 (3 s), 130.3 (d), 129.0 (s), 128.5, 128.2, 126.3, 113.9, 80.7 (5 d), 55.2 (q), 41.5, 39.9, 32.6, 29.4 (4 t).

### 5-(4-fluorobenzyl)-3-phenethyl-4,5-dihydroisoxazole (66)

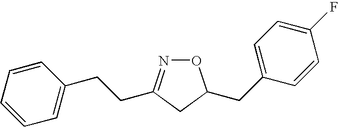

Yield 30%, a pale yellow viscous ; ¹H NMR δ 7.28 (m, 2H), 7.22-7.16 (m, 3H), 7.14 (m, 2H), 6.96 (m, 2H), 4.72 (m, 1H), 2.89 (dd, 1H, J=14.0, 6.3), 2.86-2.78 (m, 3H), 2.74 (dd, 1H, J=14.0, 6.2), 2.65-2.51 (m, 3H); ¹³C NMR δ 161.8 (d), 158.1, 140.5 (2 s), 132.8 (d), 130.9 (dd), 128.6, 128.2, 126.3 (3 d), 115.3 (dd), 80.4 (d), 41.6, 40.0, 32.6, 29.4 (4 t).

### 5-naphthalen-1-yl-methyl-3-phenethyl-4,5-dihydroisoxazole (67)

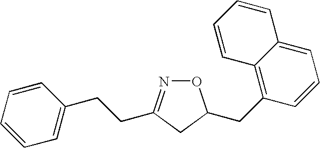

Yield 35%, a colourless wax; ¹H NMR δ 8.00 (d, 1H, J=8.4), 7.84 (dd, 1H, J=8.6, 1.4), 7.74 (d, 1H, J=8.2), 7.52-7.44 (m, 2H), 7.38 (dd, 1H, J=8.1, 7.1), 7.29-7.23 (m, 3H), 7.20-7.15 (m, 3H), 4.95 (m, 1H), 3.52 (dd, 1H, J=14.1, 5.9), 3.08 (dd, 1H, J=14.1, 7.9), 2.87-2.81 (m, 2H), 2.76 (dd, 1H, J=17.0, 10.0), 2.68-2.57 (m, 3H); ¹³C NMR δ 158.2, 140.5, 133.9, 133.1, 132.0 (5 s), 128.8, 128.5, 128.3, 127.5, 127.4, 126.3, 126.2, 125.7, 125.5, 123.6, 79.8 (11 d), 42.0, 37.9, 32.6, 29.4 (4 t).

### 5-naphthalen-2-yl-methyl-3-phenethyl-4,5-dihydroisoxazole (68)

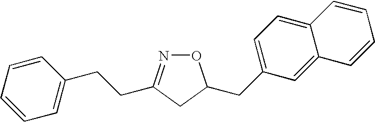

Yield 49%, a colourless wax; ¹H NMR δ 7.82-7.75 (m, 3H), 7.61 (s, 1H), 7.45 (m, 2H), 7.33 (dd, 1H, J=8.4, 1.7), 7.27 (m, 2H), 7.20 (m, 1H), 7.15 (m, 2H), 4.87 (m, 1H), 3.15 (dd, 1H, J=13.9, 6.0), 2.92 (dd, 1H, J=13.9, 7.0), 2.88-2.78 (m, 3H), 2.66-2.55 (m, 3H); ¹³C NMR δ 158.2, 140.5, 134.6, 133.5, 132.3 (5 s), 128.6, 128.3, 128.2, 127.9, 127.7, 127.6, 127.5, 126.3, 126.1, 125.6, 80.5 (11 d), 41.7, 41.0, 32.6, 29.4 (4 t).

### 3-(3-Fluorobenzyl)-5-phenethyl-4,5-dihydroisoxazole (69)

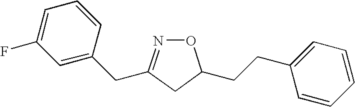

Yield 21%, a pale yellow oil; ¹H NMR δ 7.30-7.24 (m, 3H), 7.20-7.14 (m, 3H), 7.00 (d, 1H, J=7.5), 6.97-6.92 (m, 2H), 4.54 (m, 1H), 3.65 (s, 2H), 2.85 (dd, 1H, J=17.0, 10.3), 2.77-2.63 (m, 2H), 2.43 (dd, 1H, J=17.0, 8.0), 1.97 (m, 1H), 1.78 (m, 1H); ¹³C NMR δ 163.0 (d), 157.1, 141.2 (2 s), 138.4 (d), 130.4 (dd), 128.5, 126.0 (2 d), 124.5 (dd), 115.8, 114.1 (2 dd), 79.7 (d), 41.3, 36.9, 34.1, 31.8 (4 t).

### 5-[2-(3-Fluorophenyl)-ethyl]-3-(2-methoxybenzyl)-4,5-dihydroisoxazole (70)

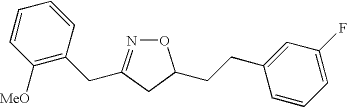

Yield 38%, a colourless viscous oil; ¹H NMR δ 7.27-7.16 (m, 3H), 6.95-6.85 (m, 5H), 4.48 (m, 1H), 3.83 (s, 3H), 3.70 (s, 2H), 2.89 (dd, 1H, J=17.1, 10.3), 2.76-2.64 (m, 2H), 2.45 (dd, 1H, J=17.1, 7.8), 1.93 (m, 1H), 1.76 (m, 1H); ¹³C NMR δ 162.9 (d), 158.1, 157.4 (2 s), 144.0 (d), 130.5 (d), 129.8 (dd), 128.5 (d), 124.4 (s), 124.1 (dd), 120.8 (d), 115.2, 112.8 (2 dd), 110.6, 79.2 (2 d), 55.4 (q), 41.8, 36.7, 31.5, 28.2 (4 t).

### 5-Methyl-3-phenethyl-5-phenyl-4,5-dihydroisoxazole (71)

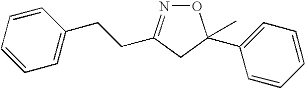

Yield 65%, yellow viscous oil, ¹H NMR δ 7.39-7.36 (m, 2H), 7.33 (m, 2H), 7.24 (m, 3H), 7.19-7.16 (1H), 7.15-7.12 (2H), 3.01 (d, 1H, J=16.8), 2.96 (d, 1H, J=16.8), 2.87 (t, 2H, J=8.0), 2.62 (t, 2H, J=8.0), 1.65 (s, 3H); ¹³C NMR δ 158.0, 145.8, 140.5 (3 s), 128.5, 128.4, 128.3, 127.2, 126.3, 124.7 (6 d), 86.8 (s), 51.0, 32.7, 29.7 (3 t), 28.1 (q).

### 3-[3-(4-Methoxy-phenyl)-propyl]-5-phenyl-4,5-dihydroisoxazole (72)

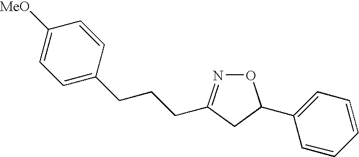

Yield 89%, yellow viscous oil, ¹H NMR δ 7.35-7.26 (m, 5H), 7.06 (m, 2H), 6.81 (m, 2H), 5.52 (dd, 1H, J=10.8, 8.0), 3.77 (s, 3H), 3.31 (ddd, 1H, J=16.9, 10.8, 0.9), 2.86 (ddd, 1H, J=16.9, 8.0, 0.9), 2.61 (t, 2H, J=7.6), 2.37 (t, 2H, J=7.7), 1.87 (tt, 2H, J=7.7, 7.6); ¹³C NMR δ 158.2, 157.9, 141.3, 133.4 (4 s), 129.4, 128.7, 128.0, 125.7, 113.9, 81.2 (6 d), 55.2 (q), 45.4, 34.3, 28.2, 27.1 (4 t).

### 5-(2-Methoxybenzyl)-3-(3-phenylpropyl)-4,5-dihydroisoxazole (73)

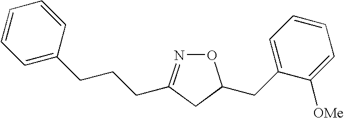

Yield 57%, a colourless oil; ¹H NMR δ 7.29-7.15 (m, 7H), 6.88 (t, 1H, J=7.4), 6.83 (d, 1H, J=8.2), 4.83 (m, 1H), 3.78 (s, 3H), 3.04 (dd, 1H, J=13.5, 5.8), 2.84-2.42 (m, 2H), 2.65-2.61 (m, 3H), 2.32 (m, 2H), 1.85 (m, 2H); ¹³C NMR δ 158.6, 157.5, 141.5 (2 s), 131.2, 128.5, 128.4, 128.3, 128.0 (5 d), 125.8 (s), 120.6, 110.4, 79.4 (3 d), 55.2 (q), 41.5, 35.4, 35.3, 28.0, 27.3 (5 t).

### 5-Benzyl-3-(2-methoxyphenyl)-5-phenyl-4,5-dihydro-isoxazole (74)

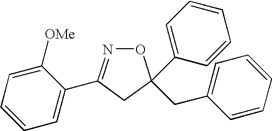

Yield 72%, mp=109.3-110.2° C., ¹H NMR δ 7.54 (dd, 1H, J=7.7, 1.7), 7.37 (m, 2H), 7.32-7.29 (m, 3H), 7.24 (m, 1H), 7.20-7.15 8m, 3H), 7.08 (m, 2H), 6.90 (ddd, 1H, J=7.5, 7.5, 1.0), 6.86 (d, 1H, J=8.4), 3.78 (s, 3H), 3.69 (d, 1H, J=17.1), 3.59 (d, 1H, J=17.1), 3.27 (d, 1H, J=13.8), 3.23 (d, 1H, J=13.8); ¹³C NMR δ 157.4, 156.2, 144.6, 136.2 (4 s), 131.1, 130.7, 129.3, 128.1, 127.8, 127.1, 126.5, 125.5, 120.7 (8 d), 119.1 (s), 111.3 (d), 90.6 (s), 55.5 (q), 48.0, 47.4 (2 t).

### 5-Benzyl-3-(2-fluorophenyl)-5-phenyl-4,5-dihydro-isoxazole (75)

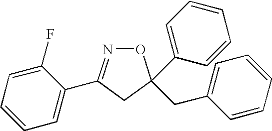

Yield 56%, mp=134.3-135.2° C., ¹H NMR δ 7.72 (ddd, 1H, J=7.7, 7.6, 1.7), 7.38 (m, 2H), 7.35-7.29 (m, 3H), 7.26 (dd, 1H, J=7.3, 7.1), 7.21-7.15 (m, 3H), 7.10 (m, 2H), 7.09 (dd, J=7.3, 1.0), 7.03 (ddd, 1H, J=10.6, 8.4, 0.8), 3.67 (dd, 1H, J=17.1, 2.4), 3.58 (dd, 1H, J=17.1, 2.5), 3.31 (d, 1H, J=13.9), 3.24 (d, 1H, J=13.9); ¹³C NMR δ 160.2, 153.3 (2 d), 144.3, 135.8 (2 s), 131.6 (dd), 130.7 (d), 128.8 (dd), 128.3, 127.9, 127.4, 126.7, 125.4, 124.3 (6 d), 117.8 (d), 116.3 (dd), 91.1 (s), 47.4 (t), 47.1 (td).

### 5-Benzyl-3-(4-chlorophenyl)-5-phenyl-4,5-dihydro-isoxazole (76)

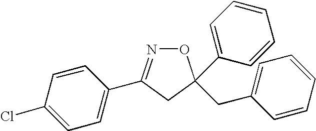

Yield 40%, mp=120.3-122.1° C., ¹H NMR δ 7.49 (m, 2H), 7.39 (m, 2H), 7.33 (m, 2H), 7.27 (m, 1H), 7.21-7.15 (m, 3H), 7.10 (m, 2H), 3.54 (d, 1H, J=16.4), 3.45 (d, 1H, J=16.4), 3.32 (d, 1H, J=14.0), 3.25 (d, 1H, J=14.0); ¹³C NMR δ 155.5, 144.2, 135.8, 135.7 (4 s), 130.6, 128.8, 128.3 (3 d), 128.2 (s), 128.0, 127.7, 127.4, 126.8, 125.3 (5 d), 91.2 (s), 47.4, 45.2 (2 t).

### 5-Benzyl-5-phenyl-3-(3-methoxyphenyl)-4,5-dihydro-isoxazole (77)

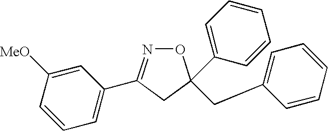

Yield 66%, mp=94.4-95.2° C., ¹H NMR δ 7.37 (m, 2H), 7.28 (m, 2H), 7.23-7.12 (m, 6H), 7.09-7.03 (m, 3H), 6.85 (d, 1H, J=7.5), 3.72 (s, 3H), 3.52 (d, 1H, J=16.4), 3.43 (d, 1H, J=16.4), 3.28 (d, 1H, J=13.9), 3.23 (d, 1H, J=13.9); ¹³C NMR δ 159.6, 156.5, 144.3, 135.8, 130.9 (5 s), 130.6, 129.5, 128.2, 127.9, 127.3, 126.7, 125.3, 119.1, 116.2, 111.0 (10 d), 90.9 (s), 55.4 (q), 47.3, 45.4 (2 t).

### Examples 78-106

### 5-Benzyl-3-(2-hydroxy-phenyl)-4,5-dihydro-isoxazole (78)

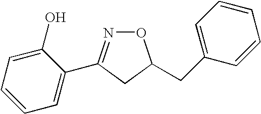

Method B: To a solution of 5-Benzyl-3-(2-methoxyphenyl)-4,5-dihydroisoxazole (compound 6, 2 mmol, synthesized using the general method described in Example 1) in 2 ml of dichloromethane was added 2.2 ml of 1M BBr₃ in dichloromethane (2.2 mmol, for compounds having two or three aromatic methoxyl groups, 4.2 mmol and 6.2 mmol was used, respectively) and the solution was stirred under argon at room temperature overnight. The organic layer was evaporated to give the oily product, which was purified by column chromatography using dichloromethane as an eluent.

Yield 99%, a brown oil, ¹H NMR (in MeOD): δ 7.25-7.18 (m, 5H), 7.16 (m, 1H), 7.11 (dd, 1H, J=7.8, 1.5), 6.90 (d, 1H, J=8.3), 6.83(m, 1H), 4.82 (m, 1H), 3.29 (dd, 1H, J=16.9, 10.2), 3.05 (dd, 1H, J=16.9, 8.1), 2.95 (dd, 1H, J=14.0, 6.9), 2.83 (dd, 1H, J=14.0, 6.2); ¹³C NMR (in MeOD): δ 155.6, 153.6, 133.6 (3 s), 128.0, 125.9, 125.3, 125.0, 123.0, 116.1, 112.9 (7 d), 110.8 (s), 77.6 (d), 37.0, 36.0 (2 t).

The following compounds included in the invention were prepared by Method B using appropriate starting materials:

### 5-Benzyl-3-(3-hydroxy-phenyl)-4,5-dihydro-isoxazole (79)

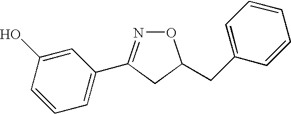

Yield 86%, a dark brown viscous oil, ¹H NMR (in MeOD): δ 7.28-7.22 (m, 4H), 7.19 (m, 2H), 7.08 (m, 1H), 7.04 (d, 1H, J=7.7), 6.85 (d, 1H, J=8.2), 4.91 (m, 1H), 3.30 (dd, 1H, J=17.0, 10.3), 3.02 (dd, 1H, J=17.0, 8.0), 3.00 (dd, 1H, J=13.9, 6.8), 2.88 (dd, 1H, J=13.9, 6.1); ¹³C NMR (in MeOD): δ 158.6, 158.5, 138.4, 131.8 (4 s), 130.8, 130.4, 129.4, 127.5, 119.1, 118.3, 114.1, 83.0 (8 d), 41.8, 40.3 (2 t).

### 5-Benzyl-3-(4-hydroxy-phenyl)-4,5-dihydro-isoxazole (80)

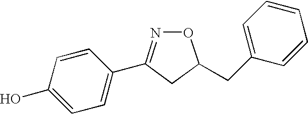

Yield 90%, a dark brown wax, ¹H NMR δ 7.48 (m, 2H), 7.32 (m, 2H), 7.26-7.22 (m, 3H), 6.84 (m, 2H), 4.94 (m, 1H), 3.28 (dd, 1H, J=16.5, 10.1), 3.15 (dd, 1H, J=13.8, 6.1), 3.04 (dd, 1H, J=16.5, 7.8), 2.88 (dd, 1H, J=13.8, 7.2.

### 5-(4-Hydroxy-benzyl)-3-(2-hydroxy-phenyl)-4,5-dihydro-isoxazole (81)

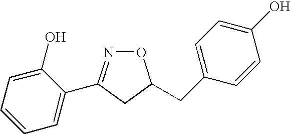

Yield 90%, a dark brown viscous oil, ¹NMR (in MeOD) δ 7.27 (m, 1H), 7.24 (d, 1H, J=7.9), 7.09 (m, 2H), 6.92 (d, 1H, J=8.2), 6.89 (m, 1H), 6.73 (m, 2H), 4.87 (m, 1H), 3.43 (dd, 1H, J=16.9, 10.2), 3.17 (dd, 1H, J=16.9, 7.9), 2.95 (dd, 1H, J=14.1, 6.4), 2.84 (dd, 1H, J=14.1, 6.4); ¹³C NMR (in MeOD) δ 160.1, 158.2, 157.1, (3 s), 132.4, 131.4, 129.8 (3 d), 128.9 (s), 120.6, 117.3, 116.3 (3 d), 115.5 (s), 82.5 (d), 40.7, 40.4 (2 t).

### 3-Benzyl-5-(3-hydroxy-phenyl)-4,5-dihydro-isoxazole (82)

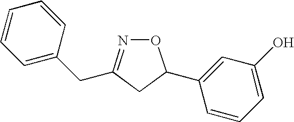

Yield 99%, a brown viscous oil, ¹NMR δ 7.34-7.28 (m, 2H), 7.25 (m, 1H), 7.21 (m, 2H), 7.14 (m, 1H), 6.79-6.74 (m, 2H), 6.72 (d, 1H, J=7.6), 5.45 (dd, 1H, J=10.8, 8.2), 3.90 (br s, 1H, OH), 3.72 (d, 1H, J=15.1), 3.66 (d, 1H, J=15.1), 3.20 (dd, 1H, J=17.3, 10.8), 2.79 (dd, 1H, J=17.3, 8.2); ¹³C NMR δ 158.1, 157.0, 142.5, 135.5 (4 s), 129.9, 128.9, 128.8, 127.2, 117.1, 115.3, 112.5, 81.8 (8 d), 44.5, 34.1 (2 t).

### 3-(4-Hydroxy-benzyl)-5-(3-hydroxy-phenyl)-4,5-dihydro-isoxazole (83)

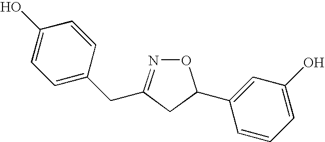

Yield 66%, a pale brown oil, ¹NMR δ 7.13 (t, 1H, J=7.6), 7.01 (m, 2H), 6.79-6.73 (m, 4H), 6.72 (d, 1H, J=7.7), 5.43 (dd, 1H, J=10.8, 8.1), 4.24 (br s, 2H, OH), 3.59 (d, 1H, J=15.0), 3.56 (d, 1H, J=15.0), 3.19 (dd, 1H, J=17.3, 10.8), 2.78 (dd, 1H, J=17.3, 8.1); ¹³C NMR δ 159.0, 156.9, 155.7, 142.4 (4 s), 129.9, 129.9 (2 d), 126.4 (s), 117.2, 115.8, 115.3, 112.5, 81.7 (5 d), 44.5, 33.2 (2 t).

### 3-(4-Hydroxy-benzyl)-5-phenyl-4,5-dihydro-isoxazole (84)

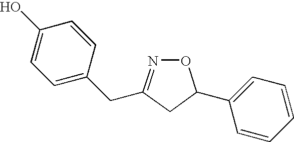

Yield 99%, a brown viscous oil, ¹H NMR δ 7.34-7.24 (m, 5H), 7.03 (m, 2H), 6.79 (m, 2H), 5.49 (dd, 1H, J=10.8, 8.5), 4.64 (s, 1H, OH), 3.63 (d, 1H, J=14.9), 3.58 (d, 1H, J=14.9), 3.22 (dd, 1H, J=17.3, 10.8), 2.81 (dd, 1H, J=17.3, 8.5); ¹³C NMR δ 158.7, 155.8, 140.8 (3 s), 129.9, 128.7, 128.2 (3 d), 126.4 (s), 125.8, 115.8, 81.9 (3 d), 44.5, 33.2 (2 t).

### 3-[2-(2-Hydroxy-phenyl)-ethyl]-5-methyl-5-phenyl-4,5-dihydro-isoxazole (85)

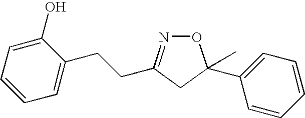

Yield 22%, brown viscous oil, ¹H NMR δ 7.39-7.32 (m, 4H), 7.26 (m, 1H), 7.09 (m, 1H), 6.70 (m, 2H), 6.60 (m, 1H), 5.56 (br s, 1H, OH), 3.03 (d, 1H, J=16.9), 2.99 (d, 1H, J=16.9), 2.81 (t, 2H, J=7.5), 2.62 (t, 2H, J=7.5), 1.66 (s, 3H); ¹³C NMR δ 158.5, 156.0, 145.6, 142.0 (4 s), 129.7, 128.5, 127.3, 124.7, 120.5, 115.3, 113.5 (7d), 87.0 (s), 51.0, 32.5, 29.4 (3 t), 28.0 (q).

### 3-[2-(3-Hydroxy-phenyl)-ethyl]-5-methyl-5-phenyl-4,5-dihydro-isoxazole (86)

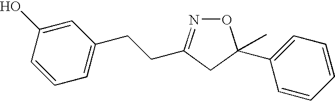

Yield 97%, brown viscous oil, ¹H NMR δ 7.37-7.31 (m, 4H), 7.25 (t, 1H, J=6.9), 7.04 (t, 1H, J=7.6), 7.00 (d, 1H, J=7.5), 6.81 (d, 1H, J=7.9), 6.75 (t, 1H, J=7.4), 4.68 (br s, 1H, OH), 3.08 (d, 1H, J=17.1), 3.06 (d, 1H, J=17.1), 2.86 (t, 2H, J=7.5), 2.65 (t, 2H, J=7.3), 1.64 (s, 3H); ¹³C NMR δ 159.8, 154.9, 145.6, (3 s), 130.2, 128.5, 127.7, 127.4 (4 d), 127.0 (s), 124.7, 120.0, 115.7 (3 d), 87.1 (s), 51.0 (t), 28.3 (q), 28.0, 27.4 (2 t).

### 3-[2-(4-Hydroxy-phenyl)-ethyl]-5-methyl-5-phenyl-4,5-dihydro-isoxazole (87)

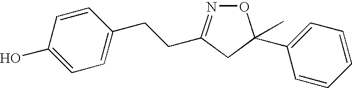

Yield 19%, brown viscous oil, ¹H NMR δ 7.38-7-32 (m, 4H), 7.26 (m, 1H), 6.96 (m, 2H), 6.69 (m, 2H), 3.03 (d, 1H, J=16.8), 2.99 (d, 1H, J=16.8), 2.79 (t, 2H, J=7.6), 2.61 (t, 2H, J=7.6), 1.65 (s, 3H); ¹³C NMR δ 158.4, 154.3, 145.6, 132.1 (4 s), 129.4, 128.5, 127.2, 124.7, 115.4 (5 d), 86.9 (s), 50.9, 31.8, 29.8 (3 t), 28.0 (q).

### 3-[3-(4-Hydroxy-phenyl)-propyl]-5-phenyl-4,5-dihydroisoxazole (88)

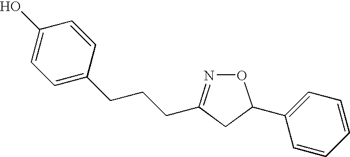

Yield 99%, a brown viscous oil, ¹H NMR δ 7.36-7.27 (m, 5H), 6.96 (m, 2H), 6.80 (m, 2H), 5.54 (dd, 1H, J=10.7, 8.3), 3.35 (dd, 1H, J=17.4, 10.7), 2.91 (dd, 1H, J=17.4, 8.3), 2.55 (t, 2H, J=7.5), 2.40 (t, 2H, J=7.6), 1.84 (tt, 2H, J=7.6, 7.5); ¹³C NMR δ 159.9, 154.5, 140.5, 132.6 (4 s), 129.5, 128.8, 128.3, 125.8, 115.7, 81.6 (6 d), 45.2, 34.3, 28.1, 27.1 (4 t).

### 3-[3-(4-Hydroxy-phenyl)-propyl]-5-(3-hydroxy-phenyl)-4,5-dihydro-isoxazole (89)

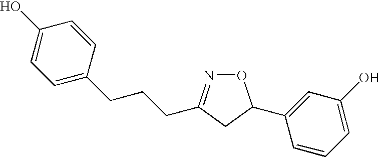

Yield 99%, a brown viscous oil, ¹H NMR δ 7.18 (m, 1H), 6.99 (m, 2H), 6.79-6.77 (m, 3H), 6.75 (m, 2H), 5.46 (dd, 1H, J=10.8, 8.0), 3.33 (dd, 1H, J=17.2, 10.8), 2.89 (dd, 1H, J=17.2, 8.0), 2.57 (t, 2H, J=7.5), 2.37 (t, 2H, J=7.6), 1.85 (tt, 2H, J=7.6, 7.5); ¹³C NMR δ 159.6, 157.4, 155.1, 142.8, 132.5 (5 s), 130.1, 129.6, 117.0, 115.5, 115.3, 112.7, 81.4 (7 d), 45.5, 34.5, 28.4, 27.2 (4 t).

### 3-[3-(4-Hydroxy-phenyl)-propyl]-5-methyl-5-phenyl-4,5-dihydro-isoxazole (90)

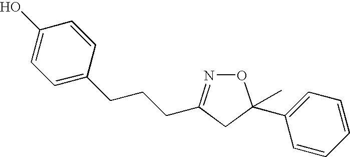

Yield 99%, a brown viscous oil, ¹H NMR δ 7.40 (m, 2H), 7.33 (m, 2H), 7.24 (m, 2H), 6.92 (m, 2H), 6.75 (m, 2H), 6.54 (br s, 1H, OH), 3.06 (d, 1H, J=16.9), 3.02 (d, 1H, J=16.9), 2.49 (m, 2H), 2.31 (t, 2H, J=7.6), 1.79 (m, 2H), 1.69 (s, 3H); ¹³C NMR δ 159.3, 154.3, 145.5, 132.9 (4 s), 129.4, 128.5, 127.3, 124.6, 115.4 (5 d), 86.7 (s), 50.7, 34.2, 28.1 (3 t), 28.0 (q), 27.3 (t).

### 5-Benzyl-3-[2-(2-hydroxy-phenyl)-ethyl]-4,5-dihydro-isoxazole (91)

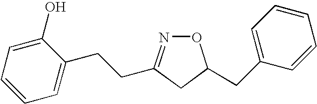

Yield 96%, a brown viscous oil, ¹H NMR δ 7.28 (m, 2H), 7.23 (m, 1H), 7.18 (m, 2H), 7.11 (m, 1H), 6.71-6.67 (m, 3H), 5.57 (br s, 1H, OH), 4.78 (m, 1H), 2.96 (dd, 1H, J=13.9, 6.0), 2.85 (dd, 1H, J=17.1, 10.1), 2.78-2.72 (m, 3H), 2.65-2.55 (m, 3H); ¹³C NMR δ 159.4, 156.4, 141.8, 136.7 (4 s), 129.7, 129.3, 128.5, 126.7, 120.1, 115.4, 113.7, 80.7 (8 d), 41.6, 40.7, 32.3, 29.0 (4 t).

### 5-Benzyl-3-[2-(3-hydroxy-phenyl)-ethyl]-4,5-dihydro-isoxazole (92)

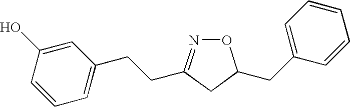

Yield 99%, mp=120.6-122.8° C., ¹H NMR δ 7.81 (br s, 1H, OH), 7.28 (m, 2H), 7.22 (m, 1H), 7.18 (m, 2H), 7.07 (m, 1H), 6.92 (d, 1H, J=7.9), 6.84 (t, 1H, J=6.9), 4.79 (m, 1H), 2.96 (dd, 1H, J=13.9, 6.3), 2.91-2.85 (m, 3H), 2.78 (dd, 1H, J=13.9, 6.5), 2.70-2.62 (m, 3H); ¹³C NMR δ 160.2, 154.5, 136.7 (3 s), 130.2, 129.3, 128.5, 127.8 (4 d), 127.2 (s), 126.7, 120.4, 116.9, 81.0 (4 d), 41.8, 40.6, 28.8, 26.6 (4 t).

### 5-Benzyl-3-[2-(4-hydroxy-phenyl)-ethyl]-4,5-dihydro-isoxazole (93)

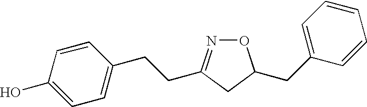

Yield 89%, mp=143.0-144.8° C., ¹H NMR δ 7.29 (m, 2H), 7.22 (m, 1H), 7.18 (m, 2H) 6.98 (m, 2H), 6.79 (m, 2H), 6.52 (br s, 1H, OH), 4.78 (m, 1H), 2.95 (dd, 1H, J=13.9, 6.1), 2.88 (dd, 1H, J=17.1, 10.2), 2.76 (dd, 1H, J=13.9, 6.7), 2.72 (m, 2H), 2.62 (dd, 1H, J=17.1, 7.5), 2.57 (m, 2H); ¹³C NMR δ 159.3, 155.4, 137.0, 131.4 (4 s), 129.5, 129.3, 128.6, 126.8, 115.5, 80.8 (6 d), 41.7, 40.9, 31.9, 29.7 (4 t).

### 5-(4-Hydroxy-benzyl)-3-phenethyl-4,5-dihydro-isoxazole (94)

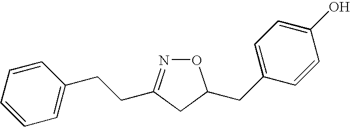

Yield 51%, mp=124.8-126.4° C., ¹H NMR δ 7.25 (m, 2H), 7.17 (m, 1H), 7.13 (m, 2H), 6.99 (m, 2H), 6.78 (m, 2H), 4.71 (m, 1H), 2.85-2.77 (m, 4H), 2.68 (dd, 1H, J=14.0, 6.4), 2.60-2.53 (m, 3H); ¹³C NMR δ 159.0, 155.1, 140.2 (3 s), 130.5, 128.5, 128.2 (3 d), 128.2 (s), 126.3, 115.6, 80.9 (3 d), 41.5, 39.8, 32.5, 29.3 (4 t).

### 5-(4-Hydroxy-benzyl)-3-[2-(2-hydroxy-phenyl)-ethyl]-4,5-dihydro-isoxazole (95) (in MeOD)

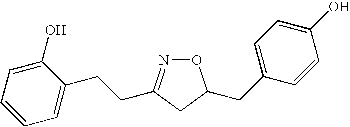

Yield 98%, a dark brown viscous oil, ¹H NMR δ 7.04-7.00 (m, 4H), 6.74 (d, 1H, J=7.6), 6.73-6.69 (m, 3H), 4.69 (m, 1H), 2.95 (dd, 1H, J=17.2, 10.1), 2.82-2.78 (m, 3H), 2.72-2.56 (m, 4H); ¹³C NMR δ 161.4, 156.9, 156.2 (3 s), 131.3, 131.0 (2 d), 129.1 (s), 128.4 (d), 127.8 (d), 120.5 (s), 116.1, 115.9, 82.3 (3 d), 42.1, 40.8, 28.6, 28.3 (4 t).

### 5-(4-Hydroxy-benzyl)-3-[2-(4-hydroxy-phenyl)-ethyl]-4,5-dihydro-isoxazole (96) (in MeOD)

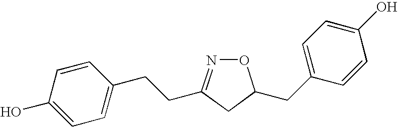

Yield 99%, a dark brown viscous oil, ¹H NMR δ 7.01 (m, 2H), 6.99 (m, 2H), 6.71 (m, 2H), 6.71 (m, 2H), 4.69 (m, 1H), 2.89 (dd, 1H, J=17.2, 10.2), 2.76 (dd, 1H, J=14.0, 6.1), 2.72-2.62 (m, 4H), 2.55 (m, 2H); ¹³C NMR δ 160.8, 157.0, 156.7, 132.5 (4 s), 131.4, 130.2 (2 d), 129.0 (s), 116.2, 116.1, 82.2 (3 d), 42.1, 40.8, 32.7, 30.4 (4 t).

### 5-(4-Fluoro-benzyl)-3-[2-(2-hydroxy-phenyl)-ethyl]-4,5-dihydro-isoxazole (97)

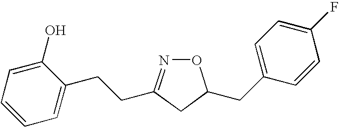

Yield 99%, mp=124.8-126.4° C., ¹H NMR δ 7.12-7.09 (m, 3H), 7.07 (d, 1H, J=7.8), 6.92 (m, 2H), 6.71-6.67 (m, 2H), 6.66 (d, 1H, J=7.7), 4.71 (m, 1H), 2.87-2.81 (m, 2H), 2.76-2.68 (m, 3H), 2.61-2.50 (m, 3H); ¹³C NMR δ 161.7 (d), 159.3, 156.4, 141.8 (3 s), 132.6 (d), 130.9 (dd), 129.7, 120.1, 115.4 (dd), 115.2, 113.6, 80.5 (3 d), 41.6, 39.9, 32.3, 28.9 (4 t).

### 5-(4-Fluoro-benzyl)-3-[2-(4-hydroxy-phenyl)-ethyl]-4,5-dihydro-isoxazole (98)

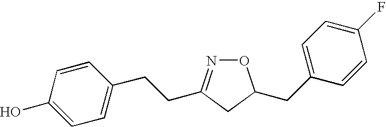

Yield 62%, a dark brown viscous oil, ¹H NMR δ 7.11 (m, 2H), 6.95-6.92 (m, 4H), 6.75 (m, 2H), 4.72 (m, 1H), 2.90-2.83 (m, 2H), 2.74-2.66 (m, 3H), 2.59-2.52 (m, 3H); ¹³C NMR δ 161.7 (d), 159.3, 154.8 (2 s), 132.5 (d), 131.5 (s), 130.9 (dd), 129.2, 115.6 (2 d), 115.3 (dd), 80.5 (d), 41.5, 39.8, 31.6, 29.3 (4 t).

### 5-Benzyl-3-[3-(4-hydroxy-phenyl)-propyl]-4,5-dihydro-isoxazole (99)

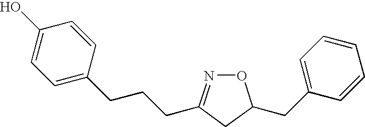

Yield 99%, a brown viscous oil, ¹H NMR δ 7.28 (m, 2H), 7.21 (m, 3H), 6.97 (m, 2H), 6.76 (m, 2H), 4.78 (m, 1H), 3.00 (dd, 1H, J=13.9, 6.0), 2.86 (dd, 1H, J=17.0, 10.1), 2.81 (dd, 1H, J=13.9, 6.9), 2.62 (dd, 1H, J=17.0, 7.6), 2.51 (t, 2H, J=7.5), 2.28 (t, 2H, J=7.7) 1.75 (m, 2H); ¹³C NMR δ 159.3, 154.5, 136.9, 132.7 (4 s), 129.5, 129.4, 128.6, 126.7, 115.3, 80.5, (6 d), 41.5, 40.9, 34.3, 28.1, 27.2 (5 t).

### 5-(4-Hydroxy-benzyl)-3-(3-phenyl-propyl)-4,5-dihydro-isoxazole (100)

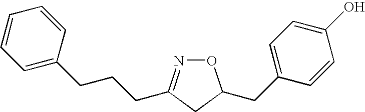

Yield 65%, a dark brown oil; ¹H NMR δ 7.24 (m, 2H), 7.15 (m, 1H), 7.11 (m, 2H), 7.00 (m, 2H), 6.80 (m, 2H), 4.71 (m, 1H), 2.87-2.81 (m, 2H), 2.72 (dd, 1H, J=14.1, 6.3), 2.60-2.54 (m, 3H), 2.28 (t, 2H, J=7.6), 1.79 (m, 2H); ¹³C NMR δ 159.5, 155.1, 141.2 (3 s), 130.4, 128.4, 128.4 (3 d), 128.1 (s), 126.0, 115.6, 80.8, (3 d), 41.3, 39.9, 35.1, 27.7, 27.2 (5 t).

### 5-(4-Hydroxy-benzyl)-3-[3-(4-hydroxy-phenyl)-propyl]-4,5-dihydro-isoxazole (101)

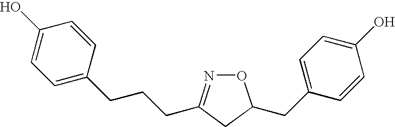

Yield 68%, a dark brown wax; ¹H NMR δ 7.02 (m, 2H), 6.95 (m, 2H), 6.77 (m, 2H), 6.76 (m, 2H), 4.73 (m, 1H), 2.86 (dd, 1H, J=14.1, 6.2), 2.85 (dd, 1H, J=17.2, 10.4), 2.74 (dd, 1H, J=14.1, 6.6), 2.62 (dd, 1H, J=17.2, 7.7), 2.50 (t, 2H, J=7.5), 2.26 (t, 2H, J=7.7) 1.73 (m, 2H); ¹³C NMR δ 159.9, 155.5, 154.8, 132.5 (4 s), 130.5, 129.5 (2d), 128.3 (s), 115.5, 115.3, 80.9 (3 d), 41.3, 39.9, 34.3, 28.1, 27.1 (5 t).

### 5-Benzyl-3-[3-(3,4-dihydroxy-phenyl)-propyl]-4,5-dihydro-isoxazole (102)

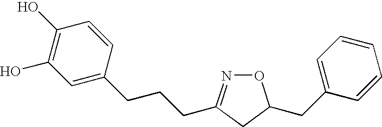

Yield 87%, a dark brown viscous oil, ¹NMR δ 7.24 (m, 2H), 7.19 (d, 1H, J=7.3), 7.17-7.14 (m, 2H), 6.76 (d, 1H, J=8.1), 6.70 (d, 1H, J=1.8), 6.61 (br s, 2H, OH), 6.47 (dd, 1H, J=8.1, 1.8), 4.75 (m, 1H), 2.94 (dd, 1H, J=13.9, 6.1), 2.83 (dd, 1H, J=17.3, 10.2), 2.77 (dd, 1H, J=13.9, 6.6), 2.58 (dd, 1H, J=17.3, 7.7), 2.38 (t, 2H, J=7.4), 2.21 (t, 2H, J=7.3), 1.68 (m, 2H); ¹³C NMR δ 160.1, 143.9, 142.2, 136.7, 133.7 (5 s), 129.4, 128.5, 126.7, 120.5, 115.7, 115.4, 80.6 (7 d), 41.4, 40.7, 34.3, 20.7, 26.9 (5 t).

### 3-[3-(3,4-Dihydroxy-phenyl)-propyl]-5-(4-hydroxy-benzyl)-4,5-dihydro-isoxazole (103) (in MeOD)

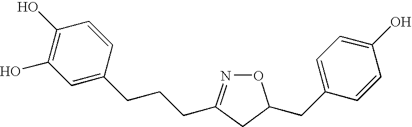

Yield 29%, a dark brown viscous oil, ¹H NMR δ 7.05 (m, 2H), 6.76 (m, 2H), 6.75 (d, 1H, J=8.0), 6.60 (d, 1H, J=2.0), 6.51 (dd, 1H, J=8.0, 2.0), 4.76 (m, 1H), 2.87-2.82 (m, 2H), 2.79 (dd, 1H, J=14.1, 6.3), 2.61 (dd, 1H, J=17.1, 7.5), 2.43 (m, 2H), 2.32 (m, 2H), 1.69 (m, 2H); ¹³C NMR δ 159.4, 155.3, 144.1, 142.7, 133.4 (5 s), 130.7 (d), 127.9 (s), 120.2, 115.5, 115.4, 115.0, 80.6 (5 d), 41.0, 39.8, 34.2, 27.8, 26.9 (5 t).

### 3[2-(2-Hydroxy-phenyl)-ethyl]-5-phenethyl-4,5-dihydro-isoxazole (104)

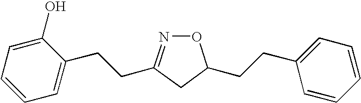

Yield 85%, a dark brown viscous oil, ¹H NMR δ 7.27 (m, 2H), 7.19 (d, 1H, J=7.3), 7.16 (m, 2H), 7.12 (dd, 1H, J=8.0, 1.7), 7.09 (d, 1H, J=7.4), 6.89 (d, 1H, J=7.7), 6.85 (t, 1H, J=7.4), 4.52 (m, 1H), 2.97-2.89 (m, 3H), 2.73 (m, 1H), 2.68-2.64 (m, 3H), 2.52 (dd, 1H, J=17.0, 8.3), 1.97 (m, 1H), 1.79 (m, 1H); ¹³C NMR δ 159.2, 154.5, 141.1 (3 s), 130.4, 128.5 (2 d), 128.4 (s), 127.9, 126.1, 120.7, 117.8, 79.7 (5 d), 42.8, 36.7, 31.8, 30.0, 25.9 (5 t).

### 3-[2-(3-Hydroxy-phenyl)-ethyl]-5-phenethyl-4,5-dihydro-isoxazole (105)

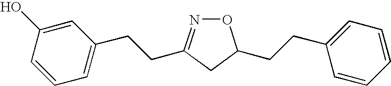

Yield 88%, a dark brown viscous oil, ¹H NMR δ 7.27 (m, 2H), 7.19 (d, 1H, J=7.3), 7.16 (m, 2H), 7.12 (t, 1H, J=8.0), 6.73-6.69 (m, 3H), 6.12 (br s, 1H, OH), 4.53 (m, 1H), 2.92 (dd, 1H, J=17.1, 10.3), 2.84 (t, 2H, J=7.6), 2.73 (ddd, 1H, J=14.2, 9.6, 5.6), 2.68-2.62 (m, 3H), 2.52 (dd, 1H, J=17.1., 7.9), 1.96 (m, 1H), 1.77 (m, 1H); ¹³C NMR δ 159.1, 156.2, 142.0, 141.1 (4 s), 129.8, 128.5, 128.5, 126.1, 120.4, 115.3, 113.6, 79.6 (8 d), 42.3, 36.8, 32.4, 31.7, 29.2 (5 t).

### 3-[2-(4-Hydroxy-phenyl)-ethyl]-5-phenethyl-4,5-dihydro-isoxazole (106)

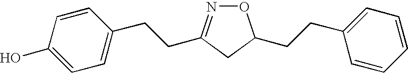

Yield 95%, a dark brown wax, ¹H NMR δ 7.28 (m, 2H), 7.20-7.17 (m, 3H), 7.03 (m, 2H), 6.74 (m, 2H), 4.50 (m, 1H), 3.02 (br s, 1H, OH), 2.90 (dd, 1H, J=16.9, 10.2), 2.80 (t, 2H, J=7.6), 2.73 (ddd, 1H, J=16.1, 10.2, 5.6), 3.04 (ddd, 1H, J=16.1, 9.5, 7.9), 2.61 (t, 2H, J=7.7), 2.50 (dd, 1H, J=16.9, 7.9), 1.95 (m, 1H), 1.77 (m, 1H).

Determination of the Estrogen Activity of the Compounds: Cell Culture, Transfection and Reporter Assays

E2 was bought from Sigma Chemical Co. (St. Louis, Mo.), and ICI-182,780 was from Tocris (Avonmouth, UK). All other reagents were of reagent grade from Sigma or Fluka. One day before transfection, HEK293 cells were seeded in 48-well plates (70×10³ cells per well) in phenol-free Dulbecco's modified Eagle medium supplemented with 5% delipidated fetal bovine serum (Sigma) and antibiotics. After a medium change, the cells were transfected for 4 hours with 5 ng ERα or ERβ expression vector, 75 ng reporter plasmid pERE₂TATA-LUC and 20 ng control plasmid pCMVβ by the calcium phosphate method. After transfection, the cells received fresh medium containing either vehicle (0.1% v/v) or test compound (10 μM). After 24 hours, the cells were washed, lysed and luciferase and β-galactosidase activities were determined from 20 μl of lysates with the Victor^{2TM} reader (PerkinElmer Wallac, Turku, Finland). After normalization for β-galactosidase activity, luciferase activities are expressed relative to that of 10 nM E2 by the formula: Activity=100%×[(Test compound)−(Vehicle)/(E2)−(Vehicle)], where terms in parenthesis indicate the corresponding normalized luciferase activities. Typically, more than 90-fold activation by 10 nM E2 of luciferase with both ER subtypes was seen. The data are means ±SEM of at least three independent transfections.

The in vitro estrogen activity of the compounds of the invention is presented in Table 1.

TABLE 1

| No. | ERα^{a} | ERβ |
|---|---|---|
| 12a^{b} | 55.2 | 17.2 |
| 12b^{c} | 9.7 | 7.9 |
| 16ab^{d} | −4.0 | 7.3 |
| 64a | 18.9 | 9.5 |
| 64b | 0.4 | 6.4 |
| 65a | 0.8 | 10.6 |
| 65b | 16.1 | 13.2 |
| 66a | 99.2 | 11.2 |
| 66b | 28.9 | 7.4 |
| 67a | 34.1 | 10.5 |
| 67b | 17.2 | 6.5 |
| 68a | 22.3 | 8.2 |
| 68b | 13.0 | 3.8 |
| 69ab | 19.5 | 10.6 |
| 69ab | 19.7 | 8.8 |
| 71a | 8.1 | −20.0 |
| 71b | 10.9 | −7.3 |
| 72a | 9.2 | −5.8 |
| 72b | 1.2 | −9.0 |
| 80a | 52.0 | 28.5 |
| 80b | 58.6 | 17.7 |
| 82ab | 21.6 | 6.6 |
| 83a | 30.8 | 33.2 |
| 83b | 3.6 | 41.3 |
| 84a | 4.8 | 0.2 |
| 84b | 7.6 | 18.5 |
| 85a | 11.8 | −0.4 |
| 85b | 21.7 | 30.0 |
| 86a | 21.6 | 0.5 |
| 86b | 7.1 | −2.9 |
| 87a | 39.7 | 18.0 |
| 87b | 18.7 | 37.4 |
| 88a | 103.6 | 44.7 |
| 88b | 65.0 | 77.1 |
| 89a | 32.9 | 12.0 |
| 89b | 27.6 | 7.2 |
| No. | ERα | ERβ |
| 90a | 88.8 | 64.8 |
| 90b | 86.5 | 30.1 |
| 91a | 104.8 | 19.2 |
| 91b | 39.7 | 30.5 |
| 92a | 19.2 | 6.7 |
| 92b | 15.7 | −2.5 |
| 93a | 171.1 | 143.1 |
| 93b | 24.3 | 41.5 |
| 94a | 34.2 | 9.1 |
| 94b | 105.2 | 42.4 |
| 95a | 20.4 | 2.4 |
| 95b | 6.2 | 0.6 |
| 96a | 76.2 | 12.0 |
| 96b | 81.4 | 4.1 |
| 97a | 82.0 | 3.4 |
| 97b | 11.1 | −2.4 |
| 98a | 77.8 | 28.4 |
| 98b | 5.7 | 4.4 |
| 99a | 153.7 | 78.2 |
| 99b | 75.1 | 19.8 |
| 100a | 67.1 | 4.7 |
| 100b | 97.2 | 17.0 |
| 101a | 14.4 | −0.9 |
| 101b | 13.6 | 10.6 |
| 102a | 0.3 | 1.3 |
| 102b | 2.1 | −1.3 |
| 103a | 1.7 | −1.1 |
| 103b | 1.4 | −0.7 |
| 104a | 4.5 | 4.2 |
| 104b | 21.8 | 2.6 |
| 105a | 8.3 | 2.6 |
| 105b | 3.0 | 3.6 |
| 106a | 62.8 | 15.4 |
| 106b | 15.8 | 0.8 |

^{a}Receptor activation (a mean of at least three independent transfections, SEM typically <15%) relative to 10 nM E2 corresponding 100, sample concentration 10 μM.
^{b}a = enantiomer with a shorter retention time in the chiral separation.
^{c}b = enantiomer with a longer retention time in the chiral separation.
^{d}ab = enantiomers not separated, rasemic mixture tested.

The in vitro activity of the compounds which are disclosed for the first time for a medical use in the present application is presented in Table 2.

TABLE 2
Previously published compounds

| No. | ERα | ERβ |
|---|---|---|
| 107a | −1.0 | 6.6 |
| 107b | 47.2 | 12.8 |
| 108ab | 37.0 | 1.2 |
| 109a | 68.4 | 1.1 |
| 109b | 38.2 | 1.3 |
| 110a | 16.4 | 9.8 |
| 110b | 11.2 | 8.1 |
| 111a | 22.8 | 8.5 |
| 111b | 1.1 | −6.0 |
| 112ab | 19.6 | 34.4 |
| 113a | 44.3 | −3.1 |
| 113b | 11.5 | 17.2 |
| 114a | 31.3 | 13.6 |
| 114b | 28.1 | 5.1 |
| 115ab | 69.5 | 15.5 |
| 116a | 3.4 | 0.2 |
| 116b | 43.9 | −0.4 |

All following compounds were synthesized according to method A.

### 5-Benzyl-3-phenyl-4,5-dihydroisoxazole (107)

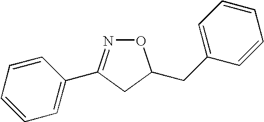

Yield 64%, mp=67.0-68.0° C., ¹H NMR δ 7.64 (m, 2H), 7.40-7.24 (m, 8H), 4.99 (m, 1H), 3.31 (dd, 1H, J=16.6, 10.2), 3.18 (dd, 1H, J=13.8, 6.1), 3.06 (dd, 1H, J=16.6, 7.8), 2.89 (dd, J=13.8, 7.3); ¹³C NMR δ 156.5, 136.9, 136.9 (3 s), 130.0, 129.4, 128.8, 128.7, 126.8, 126.6, 81.9 (7 d), 41.0, 39.4 (2 t).

### 3-Benzyl-5-phenethyl-4,5-dihydroisoxazole (108)

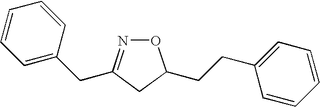

Yield 38%, a yellow viscous oil; ¹NMR δ 7.31-7.28 (m, 2H), 7.26-7.18 (m, 5H), 7.17-7.12 (m, 3H), 4.49 (m, 1H), 3.64 (s, 2H), 2.82 (dd, 1H, J=16.9, 10.3), 2.73-2.61 (m, 2H), 2.42 (dd, 1H, J=16.9, 8.0) 1.93 (m, 1H), 1.74 (m, 1H); ¹³C NMR δ 157.7, 141.2, 135.9 (3 s), 128.8, 128.8, 128.4, 128.4, 127.0, 126.0, 79.6 (7 d), 41.3, 36.9, 34.2, 31.7 (4 t).

### 5-Benzyl-3-phenethyl-4,5-dihydroisoxazole (109)

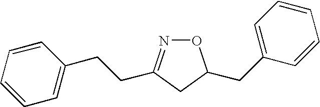

Yield 51%, a colourless oil; ¹H NMR δ 7.27-7.24 (m, 4H), 7.21-7.15 (m, 6H), 4.73 (m, 1H), 2.95 (dd, 1H, J=13.8, 6.0), 2.84-2.76 (m, 3H), 2.73 (dd, 1H, J=13.8, 6.9), 2.63-2.52 (m, 3H); ¹³C NMR δ 158.1, 140.5, 137.0 (3 s), 129.4, 128.5, 128.5, 128.2, 126.6, 126.3, 80.5 (7 d), 41.6, 40.9, 32.6, 29.4 (4 t).

### 3-Phenethyl-5-phenyl-4,5-dihydroisoxazole (110)

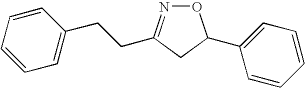

Yield 49%, mp=35.0-36.3° C.; ¹H NMR δ 7.29 (m, 2H), 7.26-7.22 (m, 5H), 7.17-7.13 (m, 3H), 5.44 (dd, 1H, J=10.8, 8.1), 3.20 (dd, 1H, J=17.0, 10.8), 2.87 (m, 2H), 2.78 (dd, 1H, J=17.0, 8.1), 2.64 (m, 2H); ¹³C NMR δ 157.7, 141.2, 140.4 (3 s), 128.6, 128.5, 128.2, 127.9, 126.3, 125.7, 81.2 (7 d), 45.4, 32.5, 29.2 (3 t).

### 5-Phenethyl-3-phenyl-4,5-dihydroisoxazole (111)

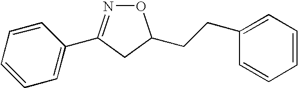

Yield 67%, mp=66.1-67.2° C.; ¹H NMR δ 7.62 (m, 2H), 7.35-7.33 (m, 3H), 7.26 (m, 2H), 7.19-7.15 (m, 3H), 4.67 (m, 1H), 3.29 (dd, 1H, J=16.5, 10.4), 2.90 (dd, 1H, J=16.5, 8.1), 2.78 (ddd, 1H, J=13.9, 9.7, 5.7), 2.74 (dd, 1H, J=13.9, 9.4, 6.8), 2.05 (m, 2H), 1.89 (m, 2H); ¹³C NMR δ 156.4, 141.1 (2 s), 129.9 (d), 129.8 (s), 128.6, 128.4, 128.4, 126.5, 126.2 , 80.2 (6 d), 39.9, 37.0, 31.8 (3 t).

### 3-Benzyl-5-phenyl-4,5-dihydroisoxazole (112)

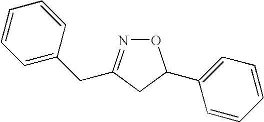

Yield 75%, a pale brown oil, ¹H NMR δ 7.35-7.26 (m, 7H), 7.25-7.22 (m, 3H), 5.52 (dd, 1H, J=10.9, 8.4), 3.74 (d, 1H, J=14.9), 3.70 (d, 1H, J=14.9), 3.22 (dd, 1H, J=17.1, 10.9), 2.80 (dd, 1H, J=17.1, 8.4); ¹³C NMR δ 157.4, 141.0, 135.7 (3 s), 128.9, 128.8, 128.7, 128.0, 127.1, 125.8, 81.9 (7 d), 44.5, 34.2 (2 t).

### 5-Phenyl-3-(3-phenyl-propyl)-4,5-dihydro-isoxazole (113)

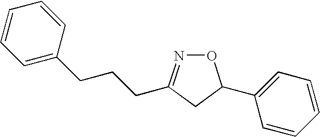

Yield 48%, a colourless oil; ¹H NMR δ 7.34-7.28 (m, 4H), 7.28-7.23 (m, 3H), 7.18-7.12 (m, 3H), 5.49 (dd, 1H, J=10.8, 8.0), 3.27 (dd, 1H, J=17.0, 10.8), 2.87 (dd, 1H, J=17.0, 8.0), 2.64 (t, 2H, J=7.6), 2.36 (t, 2H, J=7.5), 1.89 (tt, 2H, J=7.6, 7.5); ¹³C NMR δ 158.1, 141.3, 141.3 (3 s), 128.6, 128.4, 128.4, 127.9, 126.0, 125.6, 81.1 (7 d), 45.3, 35.0, 27.9, 27.1 (4 t).

### 5-Benzyl-3-(3-phenyl-propyl)-4,5-dihydro-isoxazole (114)

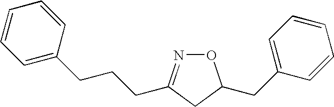

Yield 58%, a colourless oil; ¹H NMR δ 7.27-7.22 (m, 4H), 7.20-7.11 (m, 6H), 4.73 (m, 1H), 2.96 (dd, 1H, J=13.9, 6.0), 2.81-2.74 (m, 2H), 2.59-2.52 (m, 3H), 2.25 (m, 2H), 1.79 (m, 2H); ¹³C NMR δ 158.4, 141.3, 137.0 (3 s), 129.4, 128.4, 128.4, 128.3, 126.6, 125.9, 80.3 (7 d), 41.4, 40.9, 35.1, 27.8, 27.1 (5 t).

### 5-Methyl-5-phenyl-3-(3-phenyl-propyl)-4,5-dihydro-isoxazole (115)

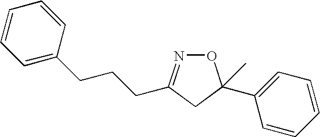

Yield 34%, yellow viscous oil, ¹H NMR δ 7.40 (m, 2H), 7.32 (m, 2H), 7.24 (m, 3H), 7.17 (m, 1H), 7.10 (m, 2H), 3.03 (d, 1H, J=16.7), 2.99 (d, 1H, J=16.7), 2.58 (m, 2H), 2.31 (t, 2H, J=7.5), 1.84 (m, 2H), 1.68 (s, 3H); ¹³C NMR δ 158.5, 145.8, 141.4 (3 s), 128.4, 128.4, 128.4, 127.2, 125.9, 124.6 (6 d), 86.6 (s), 50.7, 35.1 (2 t), 28.1 (q), 27.9, 27.4 (2 t).

### 3,5-Di-phenethyl-4,5-dihydroisoxazole (116)

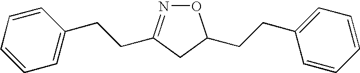

Yield 41%, mp=56.2-58.0° C.; ¹H NMR δ 7.30-7.25 (m, 4H), 7.22-7.16 (m, 6H), 4.49 (m, 1H), 2.93-2.84 (m, 3H), 2.74 (m, 1H), 2.70-2.63 (m, 3H), 2.47 (dd, 1H, J=16.8, 7.9), 1.95 (m, 1H), 1.76 (m, 1H); ¹³C NMR δ 158.1, 141.3, 140.6 (3 s), 128.5, 128.4, 128.4, 128.3, 126.3, 126.0, 79.2 (7 d), 42.4, 36.9, 32.7, 31.8, 29.6 (5 t).

Determination of Relative Binding Affinity (RBA).

Relative binding affinities (RBA) were measured by a competitive assay against [6,7-³H(N)]estradiol (PerkinElmer) in transsiently transfected COS-1 cells. One day before transfection, COS-1 cells were seeded into 2 ml of DMEM (Dulbecco's modified Eagle medium, Gipco) with 10% delipidated fetal bovine serum and 0.25% (vol/vol) Penicillin-Streptomysin (Euroclone) at a density of 140×10³ cells/well. After medium change to DMEM 2.5% FBS, the cells were transfected for 24 hours with 10 ng/well of human ERα/ERβ expression vector pSG5-hERα/β by using the TransIT method (Micrus Bio TransIT-LT1, Transfection Reagent). After 36 hours, the cells where treated with tested compunds using 0.01-, 0.1-, 1-, 10-, 100-, 1000- and 10000-fold molarities compared to labeled E2 (1,96 pM/well). After 2 hours of incubation at 37° C. the medium was removed. The cells were removed from the wells to 150 μl of 1× phosphate buffered saline (PBS), transferred to Eppendorf tubes and centrifuged at 4° C. using 4000 g for 5 min, and then washed twice with 150 μl of PBS. The cell pellets were dissolved to 50 μl of 0.5M NaOH and incubated for 15 min at 56° C., after which the samples were transferred to liquid twinkle tubes and treated with 3 ml of OptiPhase HiSafe 3 twinkle solution (PerkinElmer). The results were measured with LKB WALLAC 1214 racbeta equipment.

TABLE 3
The median inhibition concentration (IC₅₀) and relative
binding affinity (RBA) of some of the estrogen active
compounds in comparison to E2 and tamoxifen (an ER antagonist
used for the treatment of breast cancer).

| | ERα | | ERβ | |
|---|---|---|---|---|
| No. | IC₅₀ (M) | RBA (%) | IC₅₀ (M) | RBA (%) |
| E2 | 1.96 × 10⁻⁹ | 100.0 | 1.96 × 10⁻⁹ | 100.0 |
| tamoxifen | 1.30 × 10⁻⁷ | 1.511 | 9.00 × 10⁻⁸ | 2.178 |
| 88a | 1.25 × 10⁻⁸ | 15.68 | 2.05 × 10⁻⁶ | 0.096 |
| 88b | 3.00 × 10⁻⁸ | 6.533 | 1.25 × 10⁻⁶ | 0.157 |
| 90a | 2.50 × 10⁻⁷ | 0.784 | 3.10 × 10⁻⁶ | 0.063 |
| 90b | 2.30 × 10⁻⁵ | 0.009 | 2.00 × 10⁻⁵ | 0.010 |
| 93a | 2.15 × 10⁻⁷ | 0.912 | 1.25 × 10⁻⁶ | 0.157 |
| 93b | 2.15 × 10⁻⁶ | 0.091 | 1.09 × 10⁻⁶ | 0.180 |
| 113a | 1.90 × 10⁻⁶ | 0.103 | N.D.^{a} | N.D. |
| 113b | 5.60 × 10⁻⁶ | 0.035 | 5.4 × 10⁻⁶ | 0.036 |

^{a}Not detected.

Determination of the Antiestrogen Activity of the Compounds.

E2 and tamoxifen was bought from Sigma-Aldrich and ICI-182,780 from Tocris (Avonmouth, UK). One day before transfection, COS-1 cells were seeded into 1 ml of DMEM (Dulbecco's modified Eagle medium, Gibco) with 10% delipidated fetal bovine serum and 0,25% (vol/vol) Penicillin-Streptomysin (Euroclone) at a density of 70×10³ cells/well. After medium change to DMEM 2.5% FBS, the cells were transfected for 24 hours with 10 ng/well of human ERα or β expression vector pSG5-hERα/β, 100 ng/well of reporter plasmid pERE₂TATA-LUC, and 10 ng/well of control plasmid pCMV-β-gal by using the TransIT method (Micrus Bio TransIT-LT1, Transfection Reagent). After transfection, the cells received treatment with test compounds giving a final concentration of 10 μM for each compound. In the antagonist test the wells were also treated with 10⁻⁷ M estradiol. After 18 hours, the cells were washed, lysed and assayed for luciferase and normalization for β-galactosidase activities and protein concentrations. The cells were washed with 100 μl of cold phosphate buffered saline (PBS), lysed with 35 μl 1× Reporter lysis Buffer (Promega) and frosted (−70° C.) for 30 minutes. Cell lysates were placed in 1.5 ml of propylene centrifuge tubes and centrifuged with 13200 g for 5 min in room temperature. For β-galactosidase assay 10 μl of supernatant from each cell extract was transferred to a 96-well plate (96 well Elisa Microplates, PS, Microcon) and incubated for 10 min at 37° C. with 65 μl of reaction mixture consisting 0.76 gl 100×Mg-buffer, 12.5 μl ONPG (4 mg/ml), and 54.3 μl of sodium-phosphate buffer (pH 7). The reaction was stopped with treatment of 125 μl Na₂CO₃. For luciferase assay 10 μl of the supernatant was transferred to a 96-well plate (Greiner Microlon lumitrac 200) and treated with 30 μl of luciferase assay substrate solution (Promega Luciferase assay Subrate lot#23805001). For the study of protein concentrations 5 μl of the supernatant was transferred to a 96-well plate (96 well Elisa Microplates, PS, Microcon) and mixed with 200 μl of 1× Bio-rad protein assay reagent. The luciferase activities were measured with Thermo Luminoscan Ascent scanner and the protein concentrations and β-galactosidase activities with Thermo Labsystem Multiscan Ex scanner. Also blank and control samples were measured. During the maintenance, transfection, and treatment with tested compounds the cells were incubated in humidified atmosphere with 5% carbon dioxide at 37° C.

TABLE 4
The relative ERα antagonism of some of the
estrogen active compounds in comparison to tamoxifen

| No. | ERα^{a} |
|---|---|
| tamoxifen | 30.2 |
| 84a | 93.6 |
| 84b | 64.5 |
| 85a | 95.6 |
| 85b | 57.9 |
| 86a | 87.3 |
| 86b | 101.8 |
| 87a | 63.1 |
| 87b | 55.0 |
| 99ab | 132.9 |
| 101ab | 39.4 |
| 102a | 115.2 |
| 102b | 104.8 |

^{a}Receptor antagonism (a mean of at least three independent transfections, SEM typically < 15%) relative to 100 nM E2 corresponding 100 and complete antagonism corresponding to 5.7 with a sample concentration of 10 μM.

1.-18. (canceled)

19. Compounds of the formula (I) 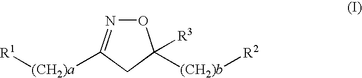
or stereoisomers, pharmaceutically acceptable salts or prodrug forms thereof,
wherein
a is an integer from 0 to 3 and b is an integer from 0 to 2, provided that a and b are not simultaneously 0 or 1;
R¹ and R² are the same or different phenyl or naphthyl groups of the formula 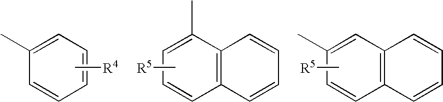
and are substituted with 0-5 R⁴ or R⁵;
R⁴ or R⁵ are the same or different and are selected from hydrogen, lower alkyl, lower alkoxyl, halogen, nitro, cyano, amino or hydroxyl;
R³ is selected from hydrogen, C₁₋₄alkyl, phenyl or benzyl;
provided that
when R³ is hydrogen, then R¹ and R² cannot simultaneously be an unsubstituted phenyl,
when a is 0, b is 1 and R³ is hydrogen, then R¹ cannot be an unsubstituted phenyl if R² is 4-hydroxy-3-methoxyphenyl, 3,4-dimethoxyphenyl, piperonyl, 1,4-dimethoxypiperonyl or 1-hydroxynapthalen-2-yl and then R¹ cannot be 4-methoxyphenyl if R² is 3,4-dimethoxyphenyl or piperonyl, and then R¹ cannot be 3,4,5-trimethoxyphenyl if R² is 3,4-dimethoxyphenyl or piperonyl, and
when a is 1, b is 0 and R³ is hydrogen, then R¹ cannot be an unsubstituted phenyl if R² is 4-methylphenyl or 4-methoxyphenyl, and
when a is 3, b is 0 and R³ is methyl, then R¹ cannot be an unsubstituted phenyl if R² also is an unsubstituted phenyl.

20. The compounds according to claim 19 having formula (I) or stereoisomers, pharmaceutically acceptable salts or prodrug forms thereof, wherein
a is 2 or 3 and b is 0 or 1,
R¹ and R² are both phenyl groups which are independently substituted by one substituent selected from the group consisting of alkoxy, halogen or hydroxyl, and
R³ is hydrogen.

21. A compound selected from the group consisting of
5-(4-fluorobenzyl)-3-phenethyl-4,5-dihydroisoxazole (66),
5-Benzyl-3-(4-hydroxy-phenyl)-4,5-dihydro-isoxazole (80),
3-(4-Hydroxy-benzyl)-5-(3-hydroxy-phenyl)-4,5-dihydro-isoxazole (83),
3-[2-(2-Hydroxy-phenyl)-ethyl]-5-methyl-5-phenyl-4,5-dihydro-isoxazole (85),
3-[2-(4-Hydroxy-phenyl)-ethyl]-5-methyl-5-phenyl-4,5-dihydro-isoxazole (87),
3-[3-(4-Hydroxy-phenyl)-propyl]-5-phenyl-4,5-dihydroisoxazole (88),
3-[3-(4-Hydroxy-phenyl)-propyl]-5-methyl-5-phenyl-4,5-dihydro-isoxazole (90),
5-Benzyl-3-[2-(2-hydroxy-phenyl)-ethyl]-4,5-dihydro-isoxazole (91),
5-Benzyl-3-[2-(4-hydroxy-phenyl)-ethyl]-4,5-dihydro-isoxazole (93),
5-(4-Hydroxy-benzyl)-3-phenethyl-4,5-dihydro-isoxazole (94),
5-(4-Hydroxy-benzyl)-3-[2-(4-hydroxy-phenyl)-ethyl]-4,5-dihydro-isoxazole (96),
5-(4-Fluoro-benzyl)-3-[2-(2-hydroxy-phenyl)-ethyl]-4,5-dihydro-isoxazole (97),
5-(4-Fluoro-benzyl)-3-[2-(4-hydroxy-phenyl)-ethyl]-4,5-dihydro-isoxazole (98),
5-Benzyl-3-[3-(4-hydroxy-phenyl)-propyl]-4,5-dihydro-isoxazole (99),
5-(4-Hydroxy-benzyl)-3-(3-phenyl-propyl)-4,5-dihydro-isoxazole (100),
3-[2-(4-Hydroxy-phenyl)-ethyl]-5-phenethyl-4,5-dihydro-isoxazole (106),
and stereoisomers, pharmaceutically acceptable salts and prodrug forms thereof.

22. The compounds according to claim 19 or 21 or stereoisomers, pharmaceutically acceptable salts or prodrugs thereof for use as pharmaceuticals.

23. A process for preparing the compounds of claim 19, comprising:
nitrile oxide—olefin cycloaddition reaction of an aldoxime comprising R¹, wherein R¹ is as defined in claim 19, with an olefin comprising R² and R³, wherein R² and R³ are as defined in claim 19, in the presence of sodium hypochlorite and pyridine to afford the desired 4,5-dihydroisoxazoles; or
demethylation reaction of the appropriate methoxy-substituted compounds in the presence of boron tribromide to afford the desired hydroxy-substituted 4,5-dihydroisoxazoles.

24. A pharmaceutical composition comprising a compound of formula (I) according to claim 19 or a stereoisomer, pharmaceutically acceptable salt or prodrug form thereof, in association with a pharmaceutically acceptable carrier.

25. A method for therapeutic or prophylactic treatment of disease states, disorders or conditions alleviated by compounds having estrogen activity, said method comprising administering an effective amount of a compound according to claim 19 having formula (I) or a stereoisomer, pharmaceutically acceptable salt or prodrug form thereof to a subject in need of such treatment.

26. The method according to claim 25 for the therapeutic or prophylactic treatment of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

27. Compounds of the formula (I) 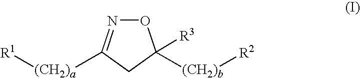
or stereoisomers, pharmaceutically acceptable salts or prodrug ns thereof,
wherein
a is an integer from 0 to 3 and b is an integer from 0 to 2, provided that a and b are not simultaneously 0 or 1;
R¹ and R² are the same or different phenyl or naphthyl groups of the formula 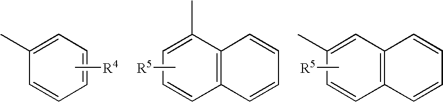
and are substituted with 0-5 R⁴ or R⁵;
R⁴ or R⁵ are the same or different and are selected from hydrogen, lower alkyl, lower alkoxyl, halogen, nitro, cyano, amino or hydroxyl;
R³ is selected from hydrogen, C₁₋₄alkyl, phenyl or benzyl,
for use as pharmaceuticals.

28. The compounds according to claim 27 or stereoisomers, pharmaceutically acceptable salts or prodrug fauns thereof for use as pharmaceuticals, wherein in the formula (I)
a is 2 or 3 and b is 0 or 1,
R¹ and R² are both phenyl groups which are independently substituted by one substituent selected from the group consisting of alkoxy, halogen or hydroxyl, and
R³ is hydrogen.

29. A pharmaceutical composition comprising a compound of the formula (I) 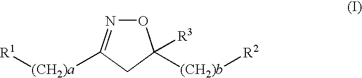
or a stereoisomer, pharmaceutically acceptable salt or prodrug form thereof,
wherein
a is an integer from 0 to 3 and b is an integer from 0 to 2, provided that a and b are not simultaneously 0 or 1;
R¹ and R² are the same or different phenyl or naphthyl groups of the formula 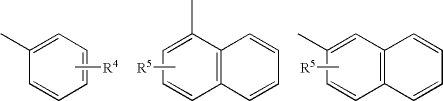
and are substituted with 0-5 R⁴ or R⁵;
R⁴ or R⁵ are the same or different and are selected from hydrogen, lower alkyl, lower alkoxyl, halogen, nitro, cyano, amino or hydroxyl;
R³ is selected from hydrogen, C₁₋₄alkyl, phenyl or benzyl,
in association with a pharmaceutically acceptable carrier.

30. A method for the therapeutic or prophylactic treatment of disease states, disorders or conditions alleviated by compounds having estrogen activity, said method comprising administering an effective amount of a compound according to claim 27 or a stereoisomer, pharmaceutically acceptable salt or prodrug form thereof to a subject in need of such treatment.

31. The method according to claim 30 for the therapeutic or prophylactic treatment of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.
